SEQUENCE LISTING (15 sequences)

SEQ ID NO: 1 - protein, 9 aa, synthetic construct
  REGION 1..9: note = PEP001
AVSSIKGAY

SEQ ID NO: 2 - protein, 5 aa, synthetic construct
  REGION 1..5: note = PEP002
AVSSI

SEQ ID NO: 3 - protein, 4 aa, synthetic construct
  REGION 1..4: note = PEP003
KGAY

SEQ ID NO: 4-5 - skipped (2 entries)

SEQ ID NO: 6 - protein, 4 aa, synthetic construct
  source 1..4: note = PEP006
AGAY

SEQ ID NO: 7 - protein, 4 aa, synthetic construct
  source 1..4: note = PEP007
RGAY

SEQ ID NO: 8 - protein, 4 aa, synthetic construct
  source 1..4: note = PEP008
IGAY

SEQ ID NO: 9 - protein, 4 aa, synthetic construct
  source 1..4: note = PEP009
SGAY

SEQ ID NO: 10 - protein, 4 aa, synthetic construct
  source 1..4: note = PEP010
VGAY

SEQ ID NO: 11-13 - skipped (3 entries)

SEQ ID NO: 14 - protein, 150 aa, synthetic construct
  REGION 1..150: note = Amuc_1409_full
MKKLHKNILFAAAVLAMPSALADIPESSVPAAAVSSIKGAYPNAGPIEWDSEMHGMERIYEAEFRINGFQFDVKIAPDGKIVRIKEEIAASSLPEAVRASVLKRYPGCRIKEAEKITEGELVRYKVEVADSRDDYDVLIAPDGNILHVDR

SEQ ID NO: 15 - protein, 127 aa, synthetic construct
  REGION 1..127: note = Amuc_1409_fragment
IPESSVPAAAVSSIKGAYPNAGPIEWDSEMHGMERIYEAEFRINGFQFDVKIAPDGKIVRIKEEIAASSLPEAVRASVLKRYPGCRIKEAEKITEGELVRYKVEVADSRDDYDVLIAPDGNILHVDR

(19) United States
(12) Patent Application Publication
(10) Pub. No.: US 2024/0209022 A1
(43) Pub. Date: Jun. 27, 2024

(54) NOVEL PEPTIDE HAVING ANTI-INFLAMMATORY AND TISSUE REGENERATIVE ACTIONS

(71) Applicant: KOREA RESEARCH INSTITUTE OF BIOSCIENCE AND BIOTECHNOLOGY, Daejeon (KR)

(72) Inventors: Chul-Ho LEE, Daejeon (KR); Yong-Hoon KIM, Daejeon (KR); Kyoung-Shim KIM, Daejeon (KR); Jung-Ran NOH, Daejeon (KR); Kyeong-Ryooon LEE, Daejeon (KR); Byoung-Chan KIM, Daejeon (KR); Dong-Hee CHOI, Daejeon (KR); Jae-Hoon KIM, Daejeon (KR); Eun-Jung KANG, Daejeon (KR); Jun GO, Daejeon (KR); Young-Keun CHOI, Daejeon (KR); In-Bok LEE, Daejeon (KR); Yun-Jung SEO, DaeJeon (KR); Jung-Hyeon CHOI, Daejeon (KR); Dong-Ho CHANG, Daejeon (KR); Hye-Yeon PARK, Daejeon (KR); Jung-Ho PARK, Daejeon (KR)

(73) Assignee: KOREA RESEARCH INSTITUTE OF BIOSCIENCE AND BIOTECHNOLOGY, Daejeon (KR)

(21) Appl. No.: 18/312,110

(22) Filed: May 4, 2023

Related U.S. Application Data
(63) Continuation-in-part of application No. PCT/KR21/15692, filed on Nov. 2, 2021.

(30) Foreign Application Priority Data
Nov. 5, 2020 (KR) 10-2020-0147160
Nov. 5, 2020 (KR) 10-2020-0147161
May 3, 2022 (KR) 10-2022-0054955

(51) Int. Cl. C07K 7/06; A61K 38/00; A61P 1/04; A61P 1/16; A61P 3/04; A61P 3/10; A61P 17/00; A61P 17/14; A61P 19/02; A61P 21/00; A61P 25/00; A61P 25/22; A61P 25/24; A61P 25/28; A61P 29/00

(52) U.S. Cl. CPC C07K 7/06; A61P 1/04; A61P 1/16; A61P 3/04; A61P 3/10; A61P 17/00; A61P 17/14; A61P 19/02; A61P 21/00; A61P 25/00; A61P 25/22; A61P 25/24; A61P 25/28; A61P 29/00; A61K 38/00

(57) ABSTRACT

The present invention relates to novel peptides having anti-inflammatory and tissue regenerative actions and uses thereof.

### CROSS REFERENCE TO RELATED APPLICATIONS

This application is a Bypass Continuation-in-Part Application of PCT/KR2021/015692 filed Nov. 2, 2021, which claims priorities based on Korean Patent Application No. 10-2020-0147160 filed Nov. 5, 2020 and Korean Patent Application No. 10-2020-0147161 filed Nov. 5, 2020, of which entire contents are incorporated by reference.

### INCORPORATION BY REFERENCE OF SEQUENCE LISTING

The content of the electronically submitted sequence listing, file name: Q287529_Sequence_Listing_As_Filed; size: 13,618 bytes; and date of creation: May 3, 2023, filed May 4, 2023, is incorporated herein by reference in its entirety.

### TECHNICAL FIELD

The present invention relates to novel peptides having anti-inflammatory and tissue regenerative actions and uses thereof.

### BACKGROUND ART

Inflammation is a complex biological response of the immune system to eliminate harmful exogenous and endogenous signals or pathogens and initiate healing processes. Various infection factors such as bacteria and viruses, ultraviolet light, heavy metals, cholesterol, asbestos, and many nanomaterials cause inflammation. The inflammatory response is essential for recovery from infection or wound healing, but uncontrolled inflammatory response or chronic inflammation may cause not only diseases such as arthritis, metabolic diseases, hepatitis, enteritis, gastritis, gastric ulcer, esophagitis, and dermatitis, but also encephalitis, depression, anxiety disorder, cognitive disorder, memory disorder, degenerative brain disease, and developmental disability.

Meanwhile, the inflammatory response often causes the function deterioration of cells, tissues, and organs to become one of the causes of damage.

For example, diseases such as skin aging, loss of muscle strength, and hair loss are diseases caused by tissue damage, and attention has recently been focused on the development of therapeutic agents capable of effectively inhibiting such tissue damage.

### PRIOR ART LITERATURE

### Patent Literature

WO 2014-200651
WO 2020-127904

### DISCLOSURE

### Technical Problem

The present inventors have conducted intensive studies to develop a therapeutic agent capable of effectively suppressing the onset and exacerbation of inflammatory diseases and tissue damage-related diseases, which have recently been increasing in modern people, as a result, developed peptides of the present invention, and confirmed that the peptides have excellent anti-inflammatory and tissue regeneration effects, thereby completing the present invention.

### Technical Solution

An object of the present invention is to provide a peptide containing a sequence represented by the following Formula 1 or 2:

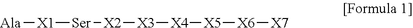

where, X1 is an amino acid classified as a nonpolar, basic, or polar amino acid;
X2 is Ser or absent;
X3 is lie or absent;
X4 is Lys or absent;
X5 is Gly or absent;
X6 is Ala or absent; and
X7 is Tyr or absent,

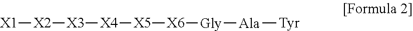

where X1 is Ala or absent;
X2 is Val or absent;
X3 is Ser or absent;
X4 is Ser or absent;
X5 is lie or absent; and
X6 is an amino acid classified as a nonpolar, acidic, basic, or polar amino acid or absent.

In an embodiment, the peptide of the present invention contains a sequence represented by the following Formula 1 or 2:

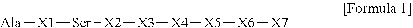

where X1 is Val, Gln, Gly, or lie;
X2 is Ser or absent;
X3 is lie or absent;
X4 is Lys or absent;
X5 is Gly or absent;
X6 is Ala or absent; and
X7 is Tyr or absent,

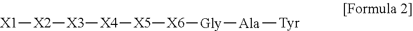

where X1 is Ala or absent;
X2 is Val or absent;
X3 is Ser or absent;
X4 is Ser or absent;
X5 is lie or absent; and
X6 is Lys, Ala, Arg, lie, Ser, Val, or absent.

Another object of the present invention is to provide a polynucleotide encoding the peptide and a vector containing the polynucleotide.

Another object of the present invention is to provide a pharmaceutical composition for preventing or treating an inflammatory disease, which contains the peptide.

Another object of the present invention is to provide a composition for tissue regeneration containing the peptide.

Another object of the present invention is to provide a pharmaceutical composition for preventing or treating a tissue damage-related disease, which contains the peptide.

Another object of the present invention is to provide a health functional food composition, a feed composition, a veterinary medicine, a quasi-drug, and a cosmetic composition, which contain the peptide.

Another object of the present invention is to provide a method for preventing or treating an inflammatory disease, which includes administering the peptide to a subject.

Another object of the present invention is to provide a tissue regeneration method including administering the peptide to a subject.

### Advantageous Effects

The peptides of the present invention have anti-inflammatory and tissue regeneration effects, and so can be used for tissue regeneration as well as prevention, improvement, and treatment of inflammatory diseases.

### BRIEF DESCRIPTION OF DRAWINGS

In the drawings of the present invention, PEP001 refers to SEQ ID NO: 1 (AVSSIKGAY), PEP002 refers to SEQ ID NO: 2 (AVSSI), PEP003 refers to SEQ ID NO: 3 (KGAY), PEP004 refers to SEQ ID NO: 4 (AVS), PEP005 refers to SEQ ID NO: 5 (GAY), PEP006 refers to SEQ ID NO: 6 (AGAY), PEP007 refers to SEQ ID NO: 7 (RGAY), PEP008 refers to SEQ ID NO: 8 (IGAY), PEP009 refers to SEQ ID NO: 9 (SGAY), PEP010 refers to SEQ ID NO: 10 (VGAY), PEP011 refers to SEQ ID NO: 11 (AQS), PEP012 refers to SEQ ID NO: 12 (AGS), PEP013 refers to SEQ ID NO: 13 (AIS), and Amuc_1409 refers to SEQ ID NO: 15, which is a functional fragment obtained by eliminating the leader sequence from the protein of SEQ ID NO: 14.

FIGS. 1a and 1b illustrate the results of administering peptides Amuc_1409 and PEP001 to PEP013 of the present invention to an animal model of degenerative arthritis and measuring and comparing knee joint thickness and weight bearing: FIG. 1a is the results of administering Amuc_1409 and PEP001 to PEP005 and measuring and comparing knee joint thickness and weight bearing (*p<0.05, **p<0.01, ***p<0.001 Amuc_1409 vs. vehicle group, ∫p<0.05, ∫∫p<0.01, ∫∫∫p<0.001 PEP001 vs. vehicle group, #p<0.05, ##p<0.01, ###p<0.001 PEP002 vs. vehicle group $p<0.05, $$$p<0.001 PEP003 vs. vehicle group, †p<0.05, ††p<0.01, †††p<0.001 PEP004 vs. vehicle group, $p<0.05, $$$p<0.001 PEP005 vs. vehicle group (Student's t-test)), FIG. 1b is the results of administering PEP006 to PEP010 and measuring and comparing knee joint thickness (A, B) and weight bearing (C, D) (***p<0.001 PEP006 vs. vehicle group, #p<0.05, ##p<0.01, ###p<0.001 PEP007 vs. vehicle group, $$p<0.01, $$$p<0.001 PEP008 vs. vehicle group, ∫p<0.05, ∫∫p<0.01, ∫∫∫p<0.001 PEP009 vs. vehicle group, †p<0.05, ††p<0.01, †††p<0.001 PEP010 vs. vehicle group (Student's t-test)), and E is the results of administering PEP011 to PEP013 and measuring and comparing weight bearing (###p<0.001 PEP0011 vs. vehicle group, $$p<0.01, $$$p<0.001 PEP0012 vs. vehicle group (Student's t-test));

FIGS. 2a and 2b illustrate the results of comparing changes in knee articular cartilage damage through Safranin O staining in an animal model of degenerative arthritis: FIG. 2a illustrates the effects acquired by administration of Amuc_1409 (**p<0.01 vs. vehicle group (Student's t-test)) and PEP001 to PEP005 (*p<0.05, **p<0.01, ***p<0.001 vs. vehicle group (Student's t-test)), and FIG. 2b illustrates the results of comparing changes in knee articular cartilage damage by administration of PEP006 to PEP010 through Safranin O staining;

FIGS. 3a to 3c illustrate the results of examining changes in arthritis index (clinical scores) by date, ankle joint thickness (bar graphs), and changes in swelling (photographs) according to the administration of Amuc_1409 and PEP001 to PEP013 in an animal model of rheumatoid arthritis: FIG. 3a illustrates the results of examining changes by administration of Amuc_1409 and PEP001 to PEP005 (p<0.05, **p<0.01 vs. vehicle group; ∫∫p<0.01, ∫∫∫p<0.001 PEP001 vs. vehicle group, ##p<0.01, ###p<0.001 PEP002 vs. vehicle group, $p<0.05, $$p<0.01, $$$p<0.001 PEP003 vs. vehicle group, †p<0.05, ††p<0.01, †††p<0.001 PEP004 vs. vehicle group, $p<0.05, $$$p<0.001 PEP005 vs. vehicle group (Student's t-test)), FIG. 3b illustrates the results of examining changes in arthritis index (clinical scores) by date, ankle joint thickness (bar graphs), and changes in swelling (photographs) by administration of PEP006 to PEP010 in an animal model of rheumatoid arthritis (*p<0.05 PEP006 vs. vehicle group, ##p<0.01 PEP007 vs. vehicle group, $p<0.05, $$p<0.01 PEP008 vs. vehicle group, ∫p<0.05, ∫∫p<0.01 PEP009 vs. vehicle group, ††p<0.01 PEP010 vs. vehicle group (Student's t-test)), and FIG. 3c illustrates the results of examining changes in arthritis index (clinical scores: A) by date, ankle joint thickness (bar graphs: B), and changes in swelling (photographs: C) by administration of PEP011 to PEP013 in an animal model of rheumatoid arthritis (#p<0.05, ##p<0.01 PEP011 vs. vehicle group, $p<0.05, $$p<0.01 PEP012 vs. vehicle group (Student's t-test)), D is the results of H&E staining by administration of PEP011 to 013 after the end of the experimental period, and E is the results showing the histopathological arthritis index (*p<0.05 vs. vehicle group (Student's t-test));

FIGS. 4a to 4c illustrate the results of examining the histopathological changes according to the administration of Amuc_1409 and PEP001 to PEP013 in an animal model of rheumatoid arthritis: FIG. 4a illustrates H&E staining results (A) and changes in histopathological arthritis index (B) by administration of Amuc_1409 and PEP001 to PEP005 after the end of the experimental period in an animal model of rheumatoid arthritis for comparison (*p<0.05, **p<0.01 vs. vehicle group (Student's t-test)), FIG. 4b illustrates H&E staining results (A, C) and changes in histopathological arthritis index (B, D) by administration of PEP006 to PEP010 after the end of the experimental period in an animal model of rheumatoid arthritis for comparison (**p<0.01, ***p<0.001 vs. vehicle group (Student's t-test)), and FIG. 4c illustrates H&E staining results (A) and changes in histopathological arthritis index (B) by administration of PEP011 to PEP013 after the end of the experimental period in an animal model of rheumatoid arthritis for comparison (**p<0.01 vs. vehicle group (Student's t-test));

FIGS. 5a and 5b illustrate the results of examining changes in obesity and diabetes indicators according to the administration of Amuc_1409 and PEP001 to PEP013 in an animal model of obesity/diabetic metabolic disease: FIG. 5a illustrates the analysis results of body weight, abdominal fat weight, insulin resistance-related indicator and fasting blood glucose by administration of Amuc_1409 and PEP001 to PEP005 in an animal model of obesity/diabetic metabolic disease (*p<0.05 Amuc_1409, #p<0.05 PEP002, $p<0.05 PEP003, †p<0.05 PEP004, $p<0.05 PEP005 vs. vehicle group (Student's t-test)), and FIG. 5b illustrates the analysis results of insulin resistance-related indicator by administration of PEP006 to PEP013 in an animal model of obesity/diabetic metabolic disease (#p<0.05 PEP007, $p<0.05 PEP008, †p<0.05 PEP010 vs. vehicle group; *p<0.05 vs. vehicle group (Student's t-test));

FIGS. 6a and 6b illustrate the analysis results of blood biochemical indicator for lipid change according to the administration of Amuc_1409 and PEP001 to PEP013 in an animal model of obesity/diabetic metabolic disease: FIG. 6a illustrates the analysis results of changes in blood lipid-related indicators according to the administration of Amuc_1409 and PEP001 to PEP005 in an animal model of obesity/diabetic metabolic disease (*p<0.05 vs. vehicle group (Student's t-test)), and FIG. 6b illustrates the analysis results of changes in blood lipid-related indicators according to the administration of PEP006 to PEP013 in an animal model of obesity/diabetic metabolic disease (*p<0.05 vs. vehicle group (Student's t-test));

FIGS. 7a and 7b illustrate the analysis results of changes in liver damage by administration of Amuc_1409 and PEP001 to PEP010 in an animal model of acute hepatitis; FIG. 7a illustrates the analysis results of blood liver damage markers (A, C, E) and H&E staining results (B, D, F) by administration of Amuc_1409 and PEP001 to PEP005 after the end of the experimental period in an animal model of acute hepatitis for comparison (*p<0.05 vs. vehicle group (Student's t-test)), and FIG. 7b illustrates the analysis results of blood liver damage markers (A) and H&E staining results (B) by administration of PEP007 to PEP010 after the end of the experimental period in an animal model of acute hepatitis for comparison (*p<0.05 vs. vehicle group (Student's t-test));

FIGS. 8a to 8c illustrate the analysis results of changes in liver damage and intrahepatic inflammation according to the administration of Amuc_1409 and PEP001 to PEP013 in an animal model of high-fat diet-induced non-alcoholic steatohepatitis: FIG. 8a illustrates the analysis results of changes in hepatic inflammation-related gene expression according to the administration of Amuc_1409 and PEP001 to PEP005 in an animal model of high-fat diet-induced non-alcoholic steatohepatitis (*p<0.05, **p<0.01 vs. vehicle group (Student's t-test)), FIG. 8b illustrates the analysis results of liver damage and changes in intrahepatic inflammation-related gene expression according to the administration of PEP006 to PEP010 in an animal model of high-fat diet-induced non-alcoholic steatohepatitis (*p<0.05 vs. vehicle group (Student's t-test)), and FIG. 8c illustrates the analysis results of liver damage and changes in intrahepatic inflammation-related gene expression according to the administration of PEP011 to PEP013 in an animal model of high-fat diet-induced non-alcoholic steatohepatitis (*p<0.05 vs. vehicle group (Student's t-test));

FIGS. 9a to 9c illustrate the results of examining the effect acquired by peptide administration in an animal model of inflammatory bowel disease: FIG. 9a illustrates the analysis results of changes in body weight by administration of PEP002 to PEP005 in a mouse model of inflammatory bowel disease (†p<0.05, ††p<0.01 PEP004 vs. vehicle group, $$p<0.01 PEP005 vs. vehicle group (Student's t-test)), FIG. 9b illustrates the analysis results of changes in colon length by administration of PEP002 to PEP005 in a mouse model of inflammatory bowel disease (*p<0.05, **p<0.01 vs. vehicle group (Student's t-test)), and FIG. 9c illustrates the analysis results of histopathological changes in the colon by administration of PEP002 to PEP005 in a mouse model of inflammatory bowel disease (*p<0.05, **p<0.01, ***p<0.001 vs. vehicle group (Student's t-test));

FIG. 10 illustrates the analysis results of the sprouting degree of organoids by the treatment with PEP006 to PEP013 in an intestinal organoid model (*p<0.05 vs. vehicle group (Student's t-test));

FIGS. 11a to 11d illustrate the analysis results of changes in gastric mucosal damage and inflammation according to the administration of Amuc_1409 and PEP001 to PEP013 in a gastritis/gastric ulcer model: FIG. 11a illustrates the analysis results of gastric mucosal damage and inflammatory marker gene by administration of Amuc_1409 in an animal model of gastritis/gastric ulcer (*p<0.05 vs. vehicle group (Student's t-test)), FIG. 11b illustrates the analysis results of gastric mucosal damage and inflammatory marker gene by administration of PEP001 to PEP005 in a gastritis/gastric ulcer model (*p<0.05 vs. vehicle group (Student's t-test)), FIG. 11c illustrates the analysis results of gastric mucosal damage by administration of PEP006 to PEP010 in an animal model of gastritis/gastric ulcer (*p<0.05 vs. vehicle group (Student's t-test)), and FIG. 11d illustrates the analysis results of gastric mucosal damage by administration of PEP011 to PEP013 in a gastritis/gastric ulcer model (*p<0.05 vs. vehicle group (Student's t-test));

FIGS. 12a to 12 c illustrate the results of examining changes in mouse ear erythema and swelling and changes in ear thickness and ear weight according to the administration of Amuc_1409 and PEP001 to PEP013 in an animal model of dermatitis: FIG. 12a illustrates the results acquired by administration of Amuc_1409 and PEP001 to PEP005 (*p<0.05, **p<0.01, ***p<0.001 vs. vehicle group (Student's t-test)), FIG. 12b illustrates the results acquired by administration of PEP006 to PEP010 (*p<0.05 vs. vehicle group (Student's t-test)), and FIG. 12c illustrates the results acquired by administration of PEP011 to PEP013 (*p<0.05 vs. vehicle group (Student's t-test));

FIGS. 13a and 14 b illustrate the results of examining histopathological changes according to the administration of Amuc_1409 and PEP001 to PEP013 in an animal model of dermatitis: FIG. 13a illustrates the results of H&E staining of histopathological mouse ear tissue by administration of Amuc_1409 and PEP001 to PEP005 in an animal model of dermatitis, and FIG. 13b illustrates the results of H&E staining of histopathological mouse ear tissue by administration of PEP007 to PEP013 in an animal model of dermatitis;

FIGS. 14a to 14c illustrate the results of examining behavioral and genetic changes according to the administration of Amuc_1409 and PEP001 to PEP013 in an animal model of depression and anxiety: FIG. 14a illustrates the analysis results of the FST and TST immobility time, the time spent in an EPM open arm, and brain inflammation index genes by administration of Amuc_1409 in a depression and anxiety model (*p<0.05 vs. vehicle group (Student's t-test)), FIG. 14b illustrates the analysis results of the FST and TST immobility time, the time spent in an EPM open arm, and brain inflammation index genes by administration of PEP001 to PEP005 in a depression and anxiety model (*p<0.05 vs. vehicle group (Student's t-test)), FIG. 14c illustrates the analysis results of the FST and TST immobility time by administration of PEP006 to PEP013 in a depression and anxiety model (*P<0.05 vs. vehicle group (Student's t-test);

FIG. 15 is graphs illustrating the effect of improving cognitive function and memory for novel objects by administration of Amuc_1409 and PEP001 in a mouse animal model of Alzheimer's disease (A and C, novel object exploration time; B and D, novel object exploration number) (*p<0.05, **p<0.01, ***p<0.01 vs. vehicle group (Student's t-test));

FIG. 16 is graphs illustrating the effect of improving cognitive function and memory in the novel object recognition test (NORT) according to the administration of Amuc_1409 and PEP002 to PEP005 in a cognitive dysfunction model induced by LPS (A and C, novel object exploration time; B and D, novel object exploration number, *p<0.05, **p<0.01, ***p<0.01 vs. vehicle group (Student's t-test));

FIG. 17 is graphs illustrating the effect of improving cognitive function and memory in the novel object recognition test (NORT) by administration of Amuc_1409 and PEP002 to PEP005 in an animal model of natural aging (A and C, novel object exploration time; B and D, novel object exploration number), and illustrates the analysis results of spontaneous alteration in the NORT test (E) and Y-maze test (F) by administration of PEP006 to PEP013 (*p<0.05, **p<0.01, ***p<0.01 vs. vehicle group (Student's t-test));

FIG. 18 illustrates the analysis results of the time (A, C, E) and frequency (B and D) of sniffing behavior to confirm the degree of social interaction and social curiosity between two mice after the administration of Amuc_1409 and PEP001 to PEP013 to animal model mice of developmental disability (*p<0.05, **p<0.01, ***p<0.01 vs. vehicle group (Student's t-test));

FIGS. 19a and 19b are photographs and graphs for comparing the wound healing effect by date according to application in a full-thickness skin defect wound-induced animal model; FIG. 19a is photographs for comparing the wound healing effect by date and the results of measuring changes in wound size by administration of Amuc_1409 and PEP001 to PEP005 (*p<0.05, **p<0.01, ***p<0.001 Amuc_1409 vs. vehicle group, ∫p<0.05, ∫∫p<0.01 PEP001 vs. vehicle group, #p<0.05, ##p<0.01, ###p<0.001 PEP002 vs. vehicle group, $p<0.05, $$p<0.01 PEP003 vs. vehicle group, ††p<0.01, †††p<0.001 PEP004 vs. vehicle group, $$p<0.01, $$$p<0.001 PEP005 vs. vehicle group (Student's t-test)), and FIG. 19b is photographs for comparing the wound healing effect by date and the results of measuring changes in wound size by administration of PEP006 to PEP013 (*p<0.05, **p<0.01 PEP006 vs. vehicle group, #p<0.05, ##p<0.01, ###p<0.001 PEP007 vs. vehicle group, $$p<0.01, $$$p<0.001 PEP008 vs. vehicle group, ∫∫p<0.01, ∫∫∫p<0.001 PEP009 vs. vehicle group, †p<0.05, ††p<0.01, †††p<0.001 PEP010 vs. vehicle group, #p<0.05, ###p<0.001 PEP011 vs. vehicle group, ∫p<0.05 PEP013 vs. vehicle group (Student's t-test));

FIGS. 20a to 20c illustrate the results of examining the effect of improving muscle strength and muscle mass in an animal model of geriatric sarcopenia: FIG. 20a illustrates the results of examining the effect of improving muscle strength, muscle mass, and muscle atrophy by administration of Amuc_1409 in an animal model of geriatric sarcopenia (*p<0.05 vs. vehicle group (Student's t-test)), FIG. 20b illustrates the analysis results of muscle strength, muscle mass, and gene expression related to protein synthesis in muscle cells according to the administration of PEP001 in an animal model of geriatric sarcopenia (*p<0.05 vs. vehicle group (Student's t-test)), and FIG. 20c illustrates the analysis results of muscle strength and muscle mass improvement by administration of PEP006 to PEP013 in an animal model of geriatric sarcopenia (*p<0.05 vs. vehicle group (Student's t-test));

FIG. 21 illustrates the analysis results of muscle atrophy-related genes by the treatment of C2C12 myoblasts with PEP004 and PEP005 (*p<0.05 vs. vehicle group (Student's t-test));

FIGS. 22a to 22d illustrate the results of examining a hair regeneration effect in an animal model: FIG. 22a is macro photographs (A) for observing the progress of hair growth on day 21 after application of Amuc_1409 and hair growth scoring graphs (B) (*p<0.05 vs. vehicle group (Student's t-test)), FIG. 22b illustrates the results of comparing changes in H&E staining results (A), histological hair follicle number (B), hair follicle depth (C), dermis thickness (D), and subcutis thickness (E) by the application of Amuc_1409 to the skin after the end of the experimental period in the animal model (**p<0.01, ***p<0.001 vs. vehicle group (Student's t-test)), FIG. 22c illustrates the results of comparing changes in proportion in histological hair growth cycle by the application of Amuc_1409 to the skin after the end of the experimental period in an animal model (***p<0.001 vs. vehicle group (Student's t-test)), and FIG. 22d is macro photographs (A) for observing the progress of hair growth on day 28 after application of PEP006 to PEP013 and hair growth scoring graphs (B);

FIG. 23 illustrates the analysis results of gene expression related to collagen degradation and synthesis according to the treatment of human skin keratinocytes with Amuc 1409 (*p<0.05 vs. vehicle group, #p<0.05 vs. vehicle group (Student's t-test)); and

FIGS. 24a to 24d illustrate the results of examining a skin regeneration effect in an animal model of aging: FIG. 24a illustrates photographs of skin H&E staining and analysis results of epidermis thickness and hair follicle number by administration of Amuc_1409 and PEP001 in an animal model of aging (*P<0.05 vs. vehicle group (Student's t-test)), FIG. 24b illustrates photographs of skin H&E staining and analysis results of epidermis thickness and hair follicle number by administration of PEP006 in an animal model of aging (*P<0.05 vs. vehicle group (Student's t-test)), FIG. 24c illustrates photographs of skin H&E staining and analysis results of epidermis thickness and hair follicle number by administration of PEP007 to PEP010 in an animal model of aging (*P<0.05 vs. vehicle group (Student's t-test)), and FIG. 24d illustrates photographs of skin H&E staining and analysis results of epidermis thickness and hair follicle number by administration of PEP011 to PEP013 in an animal model of aging (*P<0.05 vs. vehicle group (Student's t-test)).

### BEST MODE FOR CARRYING OUT THE INVENTION

Detailed description of the present invention is as follows. Meanwhile, each description and embodiment disclosed in the present invention may also be applied to other descriptions and other embodiment, respectively. In other words, all combinations of the various elements disclosed in the present invention fall within the scope of the present invention. Further, the scope of the present invention is not limited by the specific descriptions below.

Further, those skilled in the art will recognize, or be able to ascertain using no more than routine experimentation, many equivalents to the specific embodiments of the present invention described herein. Further, these equivalents should be interpreted to fall within the scope of the present invention.

An object of the present invention is to provide a peptide containing a sequence represented by the following Formula 1 or 2:

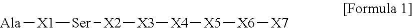

where, X1 is an amino acid classified as a nonpolar, basic, or polar amino acid;
X2 is Ser or absent;
X3 is lie or absent;
X4 is Lys or absent;
X5 is Gly or absent;
X6 is Ala or absent; and
X7 is Tyr or absent,

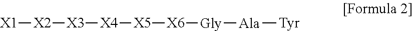

where X1 is Ala or absent;
X2 is Val or absent;
X3 is Ser or absent;
X4 is Ser or absent;
X5 is lie or absent; and
X6 is an amino acid classified as a nonpolar, acidic, basic, or polar amino acid or absent.

In an embodiment, X1 in Formula 1 may be Val, Gln, Gly, or lie.

In an embodiment, X1 in Formula 2 may be Lys, Ala, Arg, lie, Ser, Val, or absent.

In any one of the embodiments, the peptide of the present invention may contain an amino acid sequence of SEQ ID NO: 4 or 5.

In any one of the embodiments, the peptide of the present invention may contain a sequence represented by the following Formula 3:

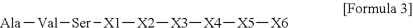

where X1 is Ser or absent;
X2 is lie or absent;
X3 is Lys or absent;
X4 is Gly or absent;
X5 is Ala or absent; and
X6 is Tyr or absent.

In any one of the embodiments, in Formula 3, X1 may be Ser, and X2 may be lie.

In any one of the embodiments, in Formula 3, X1 to X6 may be absent.

In any one of the embodiments, in Formula 3, X1 may be Ser, X2 may be lie, X3 may be Lys, X4 may be Gly, X5 may be Ala, and X6 may be Tyr.

In an embodiment, the peptide of the present invention may contain a sequence represented by the following Formula 4:

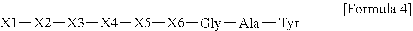

where X1 is Ala or absent;
X2 is Val or absent;
X3 is Ser or absent;
X4 is Ser or absent;
X5 is lie or absent; and
X6 is Lys or absent.

In any one of the embodiments, in Formula 4, X6 may be Lys.

In any one of the embodiments, in Formula 4, X1 to X6 may be absent.

In any one of the embodiments, in Formula 4, X1 may be Ala, X2 may be Val, X3 may be Ser, X4 may be Ser, X5 may be lie, and X6 may be Lys.

The term “absent”, for example, “X5 is lie or absent” is to be understood as amino acid residues directly adjacent to the absent amino acid are linked directly to each other by a conventional amide bond. In other words, the sequence corresponds to Ile-Gly-Ala-Tyr when X1 to X4 are absent, X5 is lie, and X6 is absent in Formula 4.

In any one of the embodiments, the peptide of the present invention may contain, consist essentially of, or consist of any one sequence selected from the following.

TABLE 1

| SEQ ID NO: | Peptide name | Sequence |
|---|---|---|
| 1 | PEP001 | AVSSIKGAY |
| 2 | PEP002 | AVSSI |
| 3 | PEP003 | KGAY |
| 4 | PEP004 | AVS |
| 5 | PEP005 | GAY |
| 6 | PEP006 | AGAY |
| 7 | PEP007 | RGAY |
| 8 | PEP008 | IGAY |
| 9 | PEP009 | SGAY |
| 10 | PEP010 | VGAY |
| 11 | PEP011 | AQS |
| 12 | PEP012 | AGS |
| 13 | PEP013 | AIS |

However, the peptide of the present invention is not limited thereto, and include peptides exhibiting the same activity as the sequences without limitation.

The peptide provided in the present invention includes its salt forms. Examples of such salts include metal salts, ammonium salts, salts with organic bases, salts with inorganic acids, salts with organic acids, salts with basic or acidic amino acids, and the like. Preferred examples of the metal salts include alkali metal salts such as a sodium salt, a potassium salt, and the like; alkaline earth metal salts such as a calcium salt, a magnesium salt, a barium salt, and the like; an aluminum salt; and the like.

In any one of the embodiments, the peptide of the present invention may contain any one of amino acid sequences of SEQ ID NOs: 1 to 15, may consist essentially of the amino acid sequence, or consist of the amino acid sequence. However, the peptide of the present invention is not limited thereto, and includes peptides exhibiting the same activity as the sequence without limitation. For example, since the amino acid sequence of SEQ ID NO: 15 of the present invention corresponds to a sequence obtained by eliminating only the leader sequence from the amino acid sequence of SEQ ID NO: 14, peptides containing, consisting essentially of, or consisting of the amino acid sequence of SEQ ID NO: 15 are also included within the scope of the peptides of the present invention.

In any one of the embodiments, the peptide of the present invention may consist of 3 to 150 consecutive amino acids in the amino acid sequence of SEQ ID NO: 14 containing the amino acid sequence of SEQ ID NO: 4 or 5.

In any one of the embodiments, the peptide of the present invention may contain, consist essentially of, or consist of any one of the amino acid sequences of SEQ ID NOs: 6 to 13.

In any one of the embodiments, the peptide of the present invention may be a peptide derived from Akkermansia muciniphila strain.

In any one of the embodiments, the peptide of the present invention can be produced by peptide synthesis methods known in the art. For example, the peptide may be one that is converted into the peptide molecule of the present invention under physiological conditions. For example, the peptide of the present invention may be synthesized through a solid phase synthesis process and a liquid phase synthesis process. In other words, the peptide provided in the present invention can be produced by repeated condensation of a partial peptide or amino acid that can constitute the peptide molecule, a peptide to be synthesized, and the remaining parts in a desired order. In a case where a product having a preferred order has a protecting group, the desired peptide can be produced by removing the protecting group.

Another aspect of the present invention provides a polynucleotide encoding the peptide.

Another aspect of the present invention provides a vector containing the polynucleotide.

The polynucleotide of the present invention is a polymer of nucleotides in which nucleotide monomers are linked in a chain shape by covalent bonds, is a DNA or RNA strand of a certain length or longer, and refers to a polynucleotide encoding the peptide according to the present invention.

In addition, in the polynucleotide of the present invention, various modifications may be made to the coding region within a range of not changing the amino acid sequence of the peptide expressed from the coding region in consideration of codons preferred in organisms to express the peptide, various modifications or transformations may be made at parts other than the coding region within a range of not affecting gene expression. In other words, as long as the polynucleotide of the present invention encodes a peptide having an activity equivalent thereto, one or more nucleic acid bases may be mutated by substitution, deletion, insertion, or a combination thereof, and these are also included in the scope of the present invention.

The recombinant vector of the present invention is introduced into a cell as a means for expressing the peptide of the present invention, known vectors such as plasmid vectors, cosmid vectors, and bacteriophage vectors may be used, and the vector may be readily prepared by those skilled in the art according to any known method using DNA recombination techniques.

Peptide analogs having functions corresponding to the peptide provided in the present invention are also included in the scope of the present invention. The term “peptide analog” refers to a non-naturally occurring amino acid sequence, or a modified naturally occurring amino acid sequence.

In any one of the embodiments, the peptide of the present invention may be in a carrier material linked form.

Some examples of carrier materials usable in the present invention may be selected from the group consisting of immunoglobulin Fc region, albumin, transferrin, and polyethylene glycol (PEG), but are not limited thereto.

PEG non-specifically binds to a specific site or various sites of a target peptide to increase the molecular weight of the peptide, thereby suppressing loss by elongation and preventing hydrolysis, and does not cause any particular side effects. In addition, when bound to a foreign peptide, PEG can sometimes inhibit the recognition of antigenic sites present in the foreign peptide by immune cells. Specifically, PEG can reduce the possibility of peptides acting as antigens by inhibiting phagocytosis and proteolysis of peptides by antigen-presenting cells.

In any one of the embodiments, the peptide of the present invention may be connected to the carrier material through a linker. The linker may be a peptide or non-peptide polymer.

Some examples of non-peptide polymers usable in the present invention may be selected from the group consisting of polyethylene glycol, polypropylene glycol, copolymer of ethylene glycol and propylene glycol, polyoxyethylated polyols, polyvinyl alcohol, polysaccharides, dextran, polyvinyl ethyl ether, biodegradable polymers such as polylactic acid (PLA) and polylactic-glycolic acid (PLGA), lipid polymers, chitins, hyaluronic acid, and combinations thereof. Derivatives of the above-mentioned exemplary non-peptide polymers already known in the art and derivatives that can be easily prepared at the level of skill in the art are also included in the scope of the present invention.

In any one of the embodiments, the peptides of the present invention may be further conjugated to moieties that may be selected from the group consisting of PEG, monosaccharides, fluorophores, chromophores, radioactive compounds and cell-penetrating peptides.

In any one of the embodiments, the peptide of the present invention may be modified by phosphorylation, sulfation, acrylation, glycosylation, methylation, farnesylation, acetylation, amidation, and the like in some cases within a range of not changing the activity of the molecule as a whole.

In any one of the embodiments, the peptide of the present invention may exist in glycosylated, PEGylated, amidated, esterified, acylated, acetylated and/or alkylated forms.

However, the peptide is not limited to the above examples.

The peptide provided in the present invention may have anti-inflammatory and/or regenerative actions.

Therefore, an aspect of the present invention provides use of the peptide of the present invention for preventing or treating an inflammatory disease.

Another aspect of the present invention provides use of the peptide of the present invention for tissue regeneration.

In the present invention, “anti-inflammation” refers to inhibiting inflammation, and the inflammation is one of the defense responses of biological tissues to certain stimuli, and refers to a complex disease that may cause tissue deterioration, circulatory disorder and exudation, and tissue proliferation.

Inflammation is part of innate immunity, and as in other animals, innate immunity in humans recognizes cell surface patterns that are specific to pathogens. Phagocytes recognize cells having such surfaces as non-self and attack the pathogen. When pathogens break through the body's physical barrier, an inflammatory response may occur. The inflammatory response is part of the immunological defense mechanism, but acute or chronic inflammatory diseases are induced when the inflammatory response occurs excessively or continuously.

Inflammation occurs in various mechanisms in the body, such as hormone secretion, cytokines, and C-reactive protein (CRP), and there are many related substances. Among these, various cytokines secreted by immune cells regulate the immune system, and some may promote inflammation. Therefore, the expression level of intracellular cytokines may be an indicator of activation of inflammatory response.

In the present invention, “inflammatory disease” means a disease caused by an inflammatory response.

The inflammatory disease may be caused by, for example, an increase in the expression of one or more inflammatory markers selected from the group consisting of Tnfα, Il-1β, Il-6, Cox2, iNos, CCI2, Cd11b, F4/80, and Ifnγ; and/or a decrease in expression of anti-inflammatory markers selected from the group consisting of Il-10 and Il-1rn. However, the inflammatory disease is not limited thereto.

The inflammatory disease of the present invention may be a disease selected from the group consisting of arthritis, metabolic diseases, hepatitis, enteritis, gastritis, gastric ulcer, esophagitis, dermatitis, encephalitis, depression, anxiety disorder, cognitive disorder, memory disorder, degenerative brain disease, and developmental disability. However, the inflammatory disease is not limited thereto.

The inflammatory disease of the present invention includes both acute and chronic diseases.

The “arthritis” of the present invention means an inflammatory disease occurring in the joints. The arthritis includes osteoarthritis, rheumatoid arthritis, chronic rheumatoid arthritis, degenerative arthritis, contusive arthritis, gouty arthritis, atrophic arthritis, chronic inflammatory arthritis, deforming arthritis, infectious arthritis, menopausal arthritis, arthritis disconnection, hypertrophic arthritis, and pyogenic arthritis.

The “metabolic disease” of the present invention is a general term for diseases caused by metabolic disorders in vivo. The metabolic disease exhibits one or more phenomena among a change in the function of the intestinal barrier, a change in the concentration of LPS in the blood, occurrence of inflammation in the intestine, a change in intestinal mucus, an increase in insulin resistance, a decrease in insulin sensitivity, an increase in fasting blood glucose, an increase in insulin resistance, an increase in glucose tolerance, and an increase in concentrations of creatinine, urea nitrogen (BUN), uric acid, and creatine kinase in the blood, or may be caused by the phenomena or a disease having the phenomena as a precursor symptom, but is not limited thereto.

The metabolic disease may be selected from the group consisting of insulin resistance disease, obesity, diabetes, fatty liver, non-alcoholic fatty liver, hyperlipidemia, kidney damage, arteriosclerosis, dyslipidemia, and hypertension, and may be selected from, for example, obesity, diabetes, insulin resistance or dyslipidemia, but is not limited thereto.

The “hepatitis” of the present invention means an inflammatory disease occurring in the liver. The hepatitis includes acute or chronic hepatitis, hepatocirrhosis, fatty liver, liver failure, liver cancer, alcoholic fatty liver disease, non-alcoholic fatty liver disease (NAFLD), non-alcoholic steatohepatitis (NASH), liver fibrosis, and liver cirrhosis. When inflammation of hepatocytes and liver tissue persists, the process of destroying and regenerating hepatocytes is repeated over a long period of time, this increases fibrous tissue and regenerative nodules in the liver, and hepatocirrhosis or liver cirrhosis may progress. For example, the hepatitis may be acute hepatitis or non-alcoholic hepatitis, but is not limited thereto. The non-alcoholic hepatitis includes, without limitation, those caused by factors other than alcohol, and may be caused by, for example, a metabolic disease such as obesity, diabetes, dyslipidemia, or metabolic syndrome, or caused by a high-fat diet, which is the cause of the metabolic disease, but is not limited thereto.

The “enteritis” of the present invention means an inflammatory disease occurring in the intestine. The “enteritis” is used in the same sense as “inflammatory bowel disease”, and includes ulcerative colitis, Crohn's disease, intestinal Bechet's disease, hemorrhagic rectal ulcer, and ileocecitis.

The “gastritis” of the present invention means an inflammatory disease occurring in the stomach. The gastritis is used as a meaning including gastric diseases such as gastric cramps, gastritis, gastric ulcer, duodenitis, and duodenal ulcer.

The “esophagitis” of the present invention means an inflammatory disease occurring in the esophagus. The esophagitis includes all of those caused by various factors such as bacterial infection, stomach contents or gastric acid reflux, and drugs, and may be, for example, reflux esophagitis, but is not limited thereto.

The “dermatitis” of the present invention means an inflammatory disease occurring in the skin. The dermatitis includes allergic contact dermatitis, urticaria, aliphatic dermatitis (dry skin on the lower legs), atopic dermatitis, contact dermatitis such as irritant contact dermatitis and poison ivy-induced contact dermatitis, eczema, gravitational dermatitis, nummular dermatitis, otitis externa, perioral dermatitis, and seborrheic dermatitis.

The “encephalitis” of the present invention means an inflammatory disease occurring in the brain. When inflammatory cells in the brain are excessively activated, the secretion of inflammatory cytokines increases, and brain cell damage may be induced by the overactivation of such brain inflammatory response. Therefore, the encephalitis has close relation with cognitive disorder, memory disorder, and degenerative brain disease. The encephalitis includes neuroinflammation.

The “depression” of the present invention refers to a disease that causes a decrease in daily function by causing various cognitive and psychosomatic symptoms with low motivation and feeling of depression as the main symptoms.

The “anxiety disorder” of the present invention is a type of psychiatric disorder, characterized by a wide range of very unpleasant and vaguely anxious feelings, accompanied by related physical symptoms (palpitations, sweating, and the like) and behavioral symptoms (irritability, restlessness, and the like). When anxious, the entire brain goes into an arousal state, so anxiety disorder interferes with peripheral behavior, autonomic nervous system, sensation, and perception. Anxiety disorder may also be associated with various combinations of psychological and physical symptoms of anxiety that are not due to actual danger and manifest as attacks (panic disorder) or persistent conditions (generalized anxiety disorder).

The “cognitive disorder” of the present invention refers to a disease caused by a decrease in cognitive function (cognitive ability) such as memory ability, time-space grasping ability, judgment, language ability or calculation ability due to brain damage. The “memory disorder” of the present invention is a pathological condition in which it is difficult or very impossible to remember things or recall past experiences, and includes diseases caused by memory decline, for example, amnesia, momentary memory loss, short-term memory loss, long-term memory loss, and transient memory disorder. However, the cognitive disorder and memory disorder are not limited thereto. For example, the peptide of the present invention may also be used to improve memory and/or cognitive ability, but is not limited thereto.

The “degenerative brain disease” of the present invention means a disease occurring in the brain among degenerative diseases. For example, the degenerative brain disease includes Alzheimer's disease, Parkinson's disease, Lou Gehrig's disease, mild cognitive disorder, stroke, and Huntington's disease. However, the degenerative brain disease is not limited thereto.

The “developmental disability” of the present invention means a state in which mental and physical development is not as developed as the age, and refers to a case in which a child is about 25% behind the normal expectation of the age in the developmental screening test. Common symptoms of developmental disability include difficulties in understanding and using language, difficulties in overall understanding of social situations and formation of interpersonal relationships, particular obsession with specific objects, and patterns of repeating certain behavioral procedures. The developmental disability in the present invention may be selected from the group consisting of intellectual disability, cerebral palsy, pervasive developmental disability, developmental language disorder, functional disorders of special senses including vision or hearing, learning disability, attention deficit hyperactivity disorder, epilepsy, and combinations thereof, but is not limited thereto. In addition, the pervasive developmental disability may be selected from the group consisting of autism spectrum disorder, Asperger's syndrome, childhood disintegrative disorder, Rett syndrome, atypical autism spectrum disorder, and combinations thereof, but is not limited thereto. In addition to these, the developmental disability may include all disabilities that occur in developmental areas such as perception, cognition, movement, and language, and may include all developmental disabilities with delays in development or function, such as learning disability, communication disorder, motor dysfunction, cerebral palsy, genetic disorder, and chromosomal disorders (Down Syndrome, Fragile X Syndrome). However, the developmental disability is not limited thereto.

An aspect of the present invention provides a pharmaceutical composition for preventing or treating an inflammatory disease, which contains the peptide provided in the present invention.

The peptide and inflammatory disease of the present invention are as described above.

In the present invention, “prevention” refers to any action that suppresses or delays the onset of a disease by administering the peptide according to the present invention. In the present invention, “treatment” refers to any action that improves or beneficially changes the symptoms of suspected or affected subjects by administering the peptide according to the present invention.

The pharmaceutical composition of the present invention may further contain suitable carriers, excipients or diluents commonly used in the preparation of pharmaceutical compositions. Specifically, the pharmaceutical composition may be formulated and used in the form of oral formulations such as powders, granules, tablets, capsules, suspensions, emulsions, syrups, and aerosols, external preparations, suppositories, and sterile injection solutions according to conventional methods, respectively. In the present invention, carriers, excipients, and diluents that may be contained in the pharmaceutical composition include lactose, dextrose, sucrose, sorbitol, mannitol, xylitol, erythritol, maltitol, starch, gum acacia, alginates, gelatin, calcium phosphate, calcium silicate, cellulose, methyl cellulose, microcrystalline cellulose, polyvinyl pyrrolidone, water, methyl hydroxybenzoate, propyl hydroxybenzoate, talc, magnesium stearate, and mineral oil. In the case of forming the pharmaceutical composition into a preparation, the preparation is prepared using diluents or excipients such as commonly used fillers, extenders, binders, wetting agents, disintegrants, and surfactants. Solid preparations for oral administration include tablets, pills, powders, granules, capsules, and the like, and these solid preparations are prepared by mixing the extract and its fractions with at least one or more excipients, such as starch, calcium carbonate, sucrose or lactose, gelatin, and the like. In addition to simple excipients, lubricants such as magnesium stearate and talc are also used. Liquid preparations for oral administration include suspensions, solutions for internal use, emulsions, and syrups, and may contain various excipients such as wetting agents, sweeteners, aromatics, and preservatives in addition to water and liquid paraffin, which are commonly used simple diluents. Preparations for parenteral administration include sterilized aqueous solutions, non-aqueous solvents, suspensions, emulsions, freeze-dried formulations, and suppositories. Propylene glycol, polyethylene glycol, vegetable oils such as olive oil, injectable esters such as ethyl oleate, and the like may be used as non-aqueous solvents and suspending agents. As a base for suppositories, witepsol, macrogol, tween 61, cacao butter, laurin, glycerogelatin, and the like may be used.

Another aspect of the present invention provides a health functional food composition for preventing or improving an inflammatory disease, which contains the peptide provided in the present invention.

Since the food composition of the present invention can be consumed on a daily basis, the effect of preventing or improving diseases can be expected, and the food composition is greatly useful.

The food composition of the present invention includes forms such as pills, powders, granules, precipitates, tablets, capsules, or liquids, and food to which the composition of the present invention can be added includes, for example, various kinds of food, such as beverages, chewing gum, tea, vitamin complexes, and health supplements.

The term “improvement” as used herein refers to any action that at least reduces the parameters related to the condition to be treated by administration of the peptide of the present invention, for example, the severity of symptoms.

The food composition of the present invention includes forms such as pills, powders, granules, precipitates, tablets, capsules, or liquids, and food to which the composition of the present invention can be added includes, for example, various kinds of food, such as beverages, chewing gum, tea, vitamin complexes, and health supplements.

There is no particular limitation on components other than the peptide provided in the present invention as an essential component that can be contained in the food composition of the present invention, and the food composition may contain various herbal extracts, food additives, or natural carbohydrates as additional components, like conventional foods. In addition, the food additives include food additives common in the art, such as flavors, flavoring agents, coloring agents, fillers, stabilizers, and the like.

Examples of the natural carbohydrates include conventional sugars such as monosaccharides, for example, glucose, fructose, and the like; disaccharides, for example, maltose, sucrose, and the like; polysaccharides, for example, dextrins and cyclodextrins, and sugar alcohols such as xylitol, sorbitol, and erythritol. In addition to the above, natural flavors (for example, rebaudioside A, glycyrrhizin, and the like) and synthetic flavors (saccharin, aspartame, and the like) may advantageously be used as flavors.

In addition to the above, the food composition of the present invention may contain various nutrients, vitamins, minerals (electrolytes), flavoring agents such as synthetic flavoring agents and natural flavoring agents, coloring agents and fillers (cheese, chocolate, and the like), pectic acid and its salts, alginic acid and its salts, organic acids, protective colloidal thickeners, pH adjusting agents, stabilizers, preservatives, glycerin, alcohol, carbonating agents used in carbonated beverages, and the like. In addition to these, the food composition may contain natural fruit juice and fruit flesh for producing fruit juice beverages and vegetable beverages. These components may be used independently or in combination.

In the present invention, the health supplement includes health functional food and health food.

The health functional food is the same term as food for special health use (FoSHU), and means food processed so that the bioregulatory function is efficiently exerted as well as nutritional supply and exhibits high medical and healthcare effects. Here, “function(al)” means to obtain useful effects for health purposes such as regulation of nutrients for the structure and function of the human body or physiological action. The food of the present invention may be prepared by a method commonly used in the art, and at the time of the preparation, may be prepared by adding raw materials and components commonly added in the art. In addition, the formulation of the food may also be prepared without limitation as long as the formulation is recognized as food. The food composition of the present invention may be prepared in various forms of formulation, has an advantage of using food as a raw material and not having side effects that may occur when drugs are taken for a long period of time unlike general drugs, and is excellent in portability, and the food of the present invention can be consumed as an adjuvant to enhance the effect of preventing or improving cognitive dysfunction and neuroinflammation.

Another aspect of the present invention provides a feed composition for preventing or improving an inflammatory disease, which contains the peptide provided in the present invention.

The feed composition may contain a feed additive. The feed additive of the present invention corresponds to supplementary feed under the Feed Management Act.

In the present invention, the term “feed” refers to any natural or artificial diet, one meal, or the like for an animal to eat, ingest, and digest or suitable therefor or a component of the one meal.

The kind of feed is not particularly limited, and feed commonly used in the art may be used. Non-limiting examples of the feed include vegetable feed such as grains, root fruits, food processing by-products, algae, fibers, pharmaceutical by-products, oils and fats, starches, cucurbits, or grain by-products and animal feed such as proteins, inorganic substances, oils and fats, mineral oils, unicellular proteins, zooplankton, or food. These may be used singly or in a combination of two or more kinds thereof.

Another aspect of the present invention provides a method for preventing or treating an inflammatory disease, which includes administering the peptide provided in the present invention to a subject.

The “subject” of the present invention means all animals such as mice, livestock, and humans that are likely to develop or have a disease. In addition, humans may be excluded from the subject of the present invention, but the subject is not limited thereto.

In the method for treating the disease of the present invention, the peptide may be administered through any general route as long as it can reach the target tissue. For example, the peptide may be administered in the form of a pharmaceutical composition.

In the treatment method of the present invention, not only a method in which the peptide provided in the present invention is administered singly, but also combination therapy may be considered. As the other agents used in the combination therapy, any one may be contained without limitation as long as it is known to have an effect of preventing or treating an inflammatory disease, and may be provided in an amount effective to produce the desired result in the prevention or treatment of inflammatory diseases. For example, the treatment method of the present invention may be achieved by administering a therapeutically effective amount of a first therapeutic agent including the peptide provided in the present invention and, optionally, a second therapeutic agent or factor. The treatment method may be accomplished by, but is not limited to, administering a single composition or pharmaceutical formulation containing both therapeutic agents or simultaneously or sequentially administering two separate compositions or formulations, one composition containing the peptide provided in the present invention and the other composition containing the second therapeutic agent.

The pharmaceutical composition of the present invention may be administered, but is not particularly limited to, through routes such as intraperitoneal administration, intravenous administration, intramuscular administration, subcutaneous administration, intradermal administration, oral administration, intranasal administration, intrapulmonary administration, and rectal administration as desired. However, since the active ingredient may be denatured by gastric acid at the time of oral administration, the oral composition may be formulated so that the active agent is coated or protected against decomposition in the stomach. For example, the pharmaceutical composition may be enteric coated so as to be protected against decomposition in the stomach, but is not limited thereto. In addition, the composition may be administered using any device through which the active substance can be transported to the target cell.

In a specific embodiment of the present invention, it has been confirmed that the administration of the peptide of the present invention inhibits inflammatory cytokine expression, increases anti-inflammatory cytokine expression, and inhibits tissue damage due to inflammation, and the peptide of the present invention can be usefully used for preventing or treating an inflammatory disease.

Another aspect of the present invention provides a veterinary medicine composition for preventing or treating an inflammatory disease, which contains the peptide provided in the present invention.

The peptide and inflammatory disease are as described above.

The animal refers to all animals such as mice, livestock, and humans that are likely to develop or have the disease, and in an embodiment, the animal may be animals except humans, but is not limited thereto.

When the composition of the present invention is used as a veterinary medicine composition, the composition may be used as it is or used together with other pharmaceutical or quasi-drug components, and may be appropriately used according to conventional methods, but is not limited thereto. The amount of the active ingredient mixed may be appropriately determined depending on the purpose of use (prevention, health, improvement, or therapeutic treatment).

Another aspect of the present invention provides a quasi-drug for preventing or improving an inflammatory disease, which contains the peptide provided in the present invention.

In the present invention, “quasi-drug” refers to items that have a milder action than pharmaceuticals among items used for the purpose of diagnosing, remedying, improving, mitigating, treating, or preventing human or animal diseases. For example, according to the Pharmaceutical Affairs Act, quasi-drugs refer to items other than those used for pharmaceutical purposes, and include products used for the treatment or prevention of human or animal diseases, products having mild or no direct action on the human body, and the like.

The quasi-drug composition of the present invention may be prepared as one or more selected from the group consisting of body cleansers, foams, soaps, masks, ointments, creams, lotions, essences, and sprays, but is not limited thereto.

Another aspect of the present invention provides a cosmetic composition for preventing or improving an inflammatory disease, which contains the peptide provided in the present invention.

The cosmetic composition may be prepared in various forms according to conventional cosmetic preparation methods. For example, the cosmetic composition may be prepared in the form of a cosmetic product, toner, cream, lotion, or the like containing the peptide of the present invention, which may be used by being diluted with a conventional cleansing liquid, astringent liquid, or moisturizing liquid.

In addition, the cosmetic composition may contain conventional adjuvants, such as stabilizers, solubilizers, vitamins, pigments, and fragrances, commonly used in the field of cosmetic compositions. In the cosmetic composition, the peptide of the present invention may be contained in an amount effective to achieve the effect of preventing or improving an inflammatory disease. The peptide of the present invention may be contained, for example, at a content of 0.001% to 10% by weight, specifically at a content of about 0.01% to 1% by weight based on the total weight of the composition, but is not limited thereto.

The formulation of the cosmetic composition may be a solution, external ointment, cream, foam, nutrient lotion, softening lotion, perfume, pack, softening water, emulsion, makeup base, essence, soap, liquid cleanser, bath additive, sunscreen cream, sun oil, suspension, emulsion, paste, gel, lotion, powder, soap, surfactant-containing cleansing, oil, powder foundation, emulsion foundation, wax foundation, patch, or spray, but is not limited thereto.

The cosmetic composition may further contain one or more cosmetically acceptable carriers that are blended in general skin cosmetics, and for example, oil, water, surfactants, humectants, lower alcohols, thickeners, chelating agents, inorganic salts, colorants, antioxidants, bactericides, preservatives, fragrances, and the like may be appropriately blended as common components, but is not limited thereto.

The peptide provided in the present invention has a regeneration effect.

Accordingly, an aspect of the present invention provides tissue regeneration use of the peptide of the present invention described above.

In an embodiment, the tissue may be selected from skin, muscle, hair, hair follicles, or hair roots.

In an embodiment, the tissue regeneration may be selected from skin regeneration, muscle tissue regeneration, wound healing, muscle strength enhancement, hair loss prevention, hair growth promotion, trichogen promotion, damaged hair improvement, and hair regeneration.

In the present invention, “regeneration” refers to suppressing deterioration of the function of cells, tissues, or organs or restoring the deteriorated function. Deterioration of the function of cells, tissues, or organs may result from cell, tissue, or organ damage. Such damage may be caused by various factors such as radiation therapy, drug therapy, surgery, infection, inflammation, degenerative disease, autoimmune disease, and aging. Meanwhile, the “regeneration” may be achieved by promoting cell differentiation, but is not limited thereto.

In an embodiment, the peptide provided in the present invention can be used for preventing or treating aging and/or geriatric diseases caused by aging.

In an embodiment, the peptide provided in the present invention may have muscle tissue regeneration ability.

Regeneration of muscle tissue may be achieved by myoblast differentiation, but is not limited thereto.

In the present invention, “myoblasts” are muscle cells in an undifferentiated state, and a skeletal muscle tissue is formed when myoblasts differentiate into skeletal muscle cells, so differentiation of myoblasts is also referred to as myogenesis. Factors involved in such differentiation of myoblasts include Mef2, serum response factor (SRF), MyoD, Myf5, Myf6, myogenin, myosin heavy chain, and the like. By measuring the expression levels of these factors, it is possible to determine whether myoblasts are differentiated.

As another example, the regeneration of muscle tissue may be achieved by inhibiting myoblast atrophy. For example, it is possible to determine whether atrophy of myoblasts is inhibited by measuring the expression levels of atrogin1 and MuRF1, which are factors involved in muscular atrophy.

Another aspect of the present invention provides a composition for tissue regeneration containing the peptide provided in the present invention.

The composition for tissue regeneration may be used for preventing or treating diseases related to tissue damage. Accordingly, another aspect of the present invention provides a pharmaceutical composition for preventing or treating a tissue damage-related disease, which contains the above-described peptide provided in the present invention as an active ingredient.

The peptide and pharmaceutical composition are as described above.

In an embodiment, the tissue of the present invention may be muscle tissue, and the tissue damage-related disease may be a muscle-related disease.

For example, the muscle-related disease may be selected from the group consisting of muscular dystrophy, muscular atrophy, muscular sarcopenia, myositis, polymyositis, peripheral vascular disease, and fibrosis, but is not limited thereto.

In an embodiment, the tissue in the present invention may be selected from hair, hair follicles, or hair roots.

The composition for tissue regeneration may exhibit effects such as hair loss prevention, hair growth promotion, trichogen promotion, damaged hair improvement, and hair regeneration. Therefore, the composition for tissue regeneration of the present invention may be used as a composition for preventing, treating, or improving hair loss. However, the composition for tissue regeneration is not limited thereto.

In an embodiment, tissue regeneration in the present invention may be wound healing. The wound healing means to treat a wound caused by damage to cells, and the wound is meant to cover all damage to a living body, and is also referred to as a cut. The wound healing may refer to any action that inhibits or delays the deterioration of a wound or improves or benefits the symptoms of a wound by administering the composition of the present invention to a wounded subject. Therefore, the composition for tissue regeneration of the present invention may be used as a composition for preventing or treating a wound or a cut.

The wound may be, for example, generated in skin tissue, but is not limited thereto.

In an embodiment, the peptide provided in the present invention may exhibit an effect of inhibiting and/or improving skin aging. Inhibition and/or improvement of skin aging may also be referred to as “skin regeneration”. Therefore, the composition for tissue regeneration of the present invention may be used as a composition for skin regeneration.

The “skin aging” of the present invention refers to the appearance of symptoms such as reduced elasticity, reduced gloss, generation of wrinkles, weakened regenerative power, or severe dryness in the skin, and may be caused by the passage of time, the external environment, or the like. The skin aging includes both endogenous aging that occurs naturally with the passage of time and photoaging that occurs in the skin by ultraviolet rays. By the skin aging of the present invention, phenomena such as decreases in the synthesized amounts of collagen, hyaluronic acid, elastin, proteoglycan fibronectin and/or precursors thereof, an increase in the expression of enzymes degrading the components, or a decrease in the expression of synthetases of the components may occur in skin cells.

The “inhibition of skin aging” of the present invention may also be referred to as “skin regeneration” and may include an increase in the amount of collagen or a precursor thereof synthesized in skin cells. The skin cells include skin keratinocytes and skin fibroblasts.

In any one of the embodiments, the inhibition of skin aging of the present invention may include an increase in collagen synthesis enzyme activity and/or inhibition of collagenase activity. For example, the collagen synthesis enzyme may be Col1a1 and/or Col3a1. For example, the collagenase may be MMP-1 and/or MMP-3. However, the inhibition of skin aging is not limited thereto.

In any one of the embodiments, the inhibition of skin aging of the present invention may include an increase in skin moisture, an increase in skin elasticity, an increase in skin thickness, improvement of skin wrinkling, alleviation of skin irritation, recovery from skin damage, and/or improvement of skin tone. However, the inhibition of skin aging is not limited thereto.

Another aspect of the present invention provides a health functional food composition for tissue regeneration, which contains the peptide provided in the present invention as an active ingredient.

The peptide, tissue regeneration, and health functional food are as described above.

Another aspect of the present invention provides a feed composition for tissue regeneration, which contains the peptide provided in the present invention as an active ingredient.

Another aspect of the present invention provides a veterinary medicine composition for tissue regeneration, which contains the peptide provided in the present invention as an active ingredient.

The peptide, tissue regeneration, feed, and veterinary medicine are as described above.

Another aspect of the present invention provides a method for preventing or treating a tissue damage-related disease, which includes administering the peptide provided in the present invention to a subject.

Another aspect of the present invention provides a tissue regeneration method including administering the peptide provided in the present invention to a subject.

In the treatment method of the present invention, not only a method in which the peptide provided in the present invention is administered singly, but also combination therapy may be considered. As the other agents used in the combination therapy, any one may be contained without limitation as long as it is known to have a tissue regeneration effect, and may be provided in an amount effective to produce the desired results in tissue regeneration.

The peptide, subject, administration, tissue damage-related disease, and prevention or treatment method are as described above.

In a specific embodiment of the present invention, it has been confirmed that the expression of genes related to tissue regeneration and differentiation due to the promotion of cell differentiation is increased by administration of the peptide of the present invention and the muscle tissue, skin tissue, hair, and hair root tissue are thus regenerated, and the peptide of the present invention can be usefully used for preventing and treating a tissue-related disease.

Another aspect of the present invention provides a quasi-drug composition for tissue regeneration, which contains the peptide provided in the present invention as an active ingredient.

Another aspect of the present invention provides a cosmetic composition for tissue regeneration, which contains the peptide provided in the present invention as an active ingredient.

The peptide, tissue regeneration, quasi-drug composition, and cosmetic composition are as described above.

### EXAMPLES

Hereinafter, the present invention will be described in more detail with reference to Examples and Experimental Examples. However, these Examples and Experimental Examples are intended to illustrate the present invention, and the scope of the present invention is not limited to these Examples and Experimental Examples.

Meanwhile, in Examples of the present invention, PEP001 refers to SEQ ID NO: 1 (AVSSIKGAY), PEP002 refers to SEQ ID NO: 2 (AVSSI), PEP003 refers to SEQ ID NO: 3 (KGAY), PEP004 refers to SEQ ID NO: 4 (AVS), PEP005 refers to SEQ ID NO: 5 (GAY), PEP006 refers to SEQ ID NO: 6 (AGAY), PEP007 refers to SEQ ID NO: 7 (RGAY), PEP008 refers to SEQ ID NO: 8 (IGAY), PEP009 refers to SEQ ID NO: 9 (SGAY), PEP010 refers to SEQ ID NO: 10 (VGAY), PEP011 refers to SEQ ID NO: 11 (AQS), PEP012 refers to SEQ ID NO: 12 (AGS), PEP013 refers to SEQ ID NO: 13 (AIS), and Amuc_1409 refers to SEQ ID NO: 15, which is a functional fragment obtained by eliminating the leader sequence from SEQ ID NO: 14.

### Example 1: Verification of Efficacy of Novel Peptides in Animal Model of Arthritis

### Example 1-1: Verification of Efficacy of Novel Peptides in Animal Model of Degenerative Arthritis

### Example 1-1-1: Experimental Method

As a degenerative arthritis model, 9-week-old specific pathogen free (SPF) male C57BL/6J mice were anesthetized, the area around the right knee joint was cleanly shaved, and 10 μL of MIA solution (monosodium iodoacetate 0.5 mg in 10 μL 0.9% sterile saline) was injected into the knee joint cavity to induce arthritis. From 3 days before MIA (monosodium iodoacetate) solution injection, Amuc_1409 or PEP001, PEP002, PEP003, PEP004, PEP005, PEP006, PEP007, PEP008, PEP009, PEP010, PEP011, PEP012, or PEP013 was dissolved in PBS buffer at a concentration of 6.7 μM and then injected into the mouse peritoneal cavity at 150 μL per animal once a day at the same time. On day 3 (0 days) after administration of Amuc_1409 or PEP001 to PEP013, an additional intraperitoneal injection was given 1 hour before MIA injection, the drug was intraperitoneally injected once a day at the same time after MIA injection, and the experiment was terminated on the 30th day after MIA injection. In the control group, the same volume of PBS buffer was injected intraperitoneally.

After MIA injection, the diameter of the knee joint was measured using a caliper for 7 days at 2-day intervals (0, 1, 3, 5, and 7 days) to evaluate swelling caused by inflammatory changes of the joint.

The induction and degree of progression of arthritis was indirectly evaluated by measuring hind limb weight bearing using an incapacitance tester at 2 weeks after MIA injection. At this time, the weight bearing on the limb was lower as arthritis was more severe. The weight-bearing weight (g) of each of both paws was measured, and in this experiment, the value was expressed as the ratio (%) of the right hind limb with arthritis to the total hind limb.

Weight bearing ratio (%)=weight-bearing weight of right hind limb with arthritis (g)/weight-bearing weight of both hind limbs (g)×100

After termination of the experiment, the test animals were sacrificed, then the knee joint tissue of the mouse was fixed with 10% neutral buffered formalin, and the bone was decalcified with a 0.5 M EDTA solution. Next, the joint tissue was embedded in paraffin to prepare a 5 μm joint section, and Safranin O staining was performed to histopathologically analyze the degree of damage to the cartilage tissue. At this time, the degree of damage to the cartilage tissue was evaluated by examining the degree of safranin O staining of the sections and scored according to OARSI (Osteoarthritis Research Society International grade) guidelines. The scoring criteria according to the OARSI guidelines are as follows (Table 1).

TABLE 1
Histopathological OARSI scoring system

| OARSI | |
|---|---|
| grade | Osteoarthritic damage |
| 0 | Normal |
| 0.5 | Decrease in Safranin O staining figure without structural change |
| 1 | Fibrillation of superficial layer without damage to cartilage |
| 2 | Vertical fragmentation of superficial zone with slight matrix loss |
| | of superficial layer |
| 3 | Vertical fissuring/erosion in area of 1% to 25% of cartilage |
| | surface |
| 4 | Vertical fissuring/erosion in area of 25% to 50% of cartilage |
| | surface |
| 5 | Vertical fissuring/erosion in area of 50% to 75% of cartilage |
| | surface |
| 6 | Vertical fissuring/erosion in area greater than 75% of cartilage |
| | surface |

### Example 1-1-2: Experimental Results

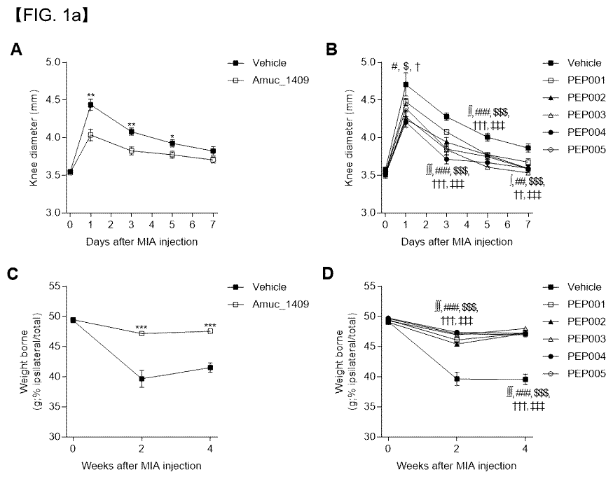

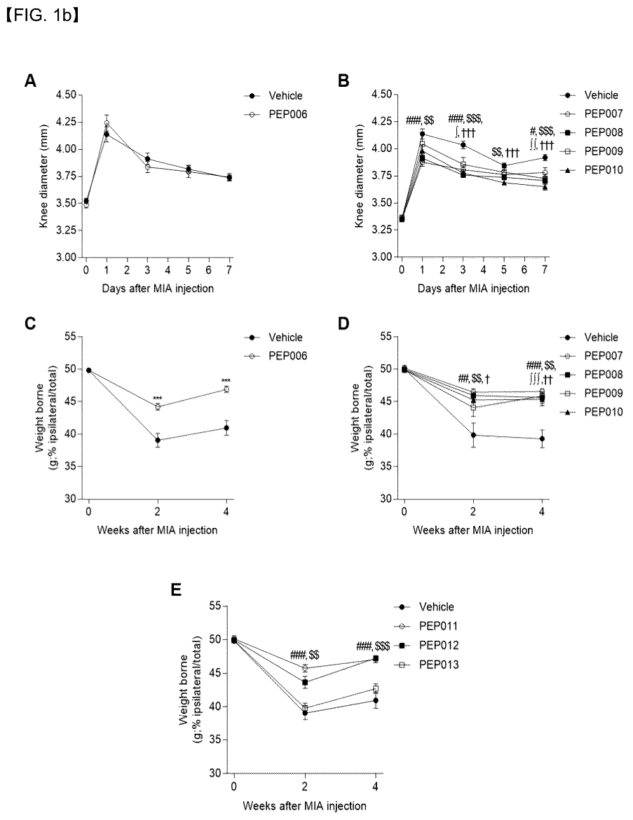

In the MIA-induced degenerative arthritis model, the diameter of the knee joint was measured using calipers for 7 days at 2-day intervals after MIA injection, and as a result, the diameter of the knee joint significantly decreased in the groups administered with Amuc_1409, PEP002, PEP003, and PEP004 and tended to decrease in the groups administered with PEP001 and PEP005 compared to the group administered with PBS buffer on day 1 after MIA injection, and the thickness of the knee joint significantly decreased in the groups administered with Amuc_1409 and PEP001, PEP002, PEP003, PEP004, and PEP005 compared to the group administered with PBS buffer on days 3, 5, and 7 after MIA injection (p<0.05, Student's t-test) (FIG. 1a).

Weight bearing was measured using an incapacitance tester 2 weeks and 4 weeks after MIA injection, and as a result, the decrease in weight bearing ratio by MIA injection significantly increased in the groups administered with Amuc_1409 and PEP001, PEP002, PEP003, PEP004, and PEP005 compared to the group administered with PBS buffer (p<0.05, Student's t-test) (FIG. 1a).

When the diameter of the knee joint was measured using calipers for 7 days at 2-day intervals after MIA injection, the diameter of the knee joint significantly decreased in the groups administered with novel tetrapeptides PEP007 and PEP008 as well and tended to decrease in the groups administered with PEP009 and PEP010 compared to the group administered with PBS buffer on day 1 after MIA injection. On day 3 after MIA injection, the thickness of the knee joint significantly decreased in the groups administered with PEP007, PEP008, PEP009, and PEP010 compared to the group administered with PBS buffer. On day 5, the thickness of the knee joint significantly decreased by administration of PEP008 and PEP010 and tended to decrease in the groups administered with PEP007 and PEP009 compared to the group administered with PBS buffer. On day 7 after MIA injection, the thickness of the knee joint significantly decreased in the groups administered with PEP007, PEP008, PEP009, and PEP010 compared to the group administered with PBS buffer (p<0.05, Student's t-test) (FIG. 1b).

In addition, weight bearing was measured using an incapacitance tester 2 weeks after MIA injection, and as a result, the decrease in weight bearing ratio by MIA injection significantly increased in the groups administered with the new tetrapeptides PEP006, PEP007, PEP008, and PEP010 and tended to increase close to a significant level in the group administered with PEP009 compared to the group administered with PBS buffer. Weight bearing was measured using an incapacitance tester 4 weeks after MIA injection, and as a result, the decrease in weight bearing ratio by MIA injection significantly increased in the groups administered with the new tetrapeptides PEP006, PEP007, PEP008, PEP009, and PEP010 compared to the group administered with PBS buffer (p<0.05, Student's t-test) (C and D in FIG. 1b). In the results of measuring weight bearing using an incapacitance tester 2 and 4 weeks after MIA injection, the decrease in weight bearing ratio by MIA injection was significantly improved in the groups administered with the novel tripeptides PEP011 and PEP012 (p<0.05, Student's t-test) and the weight bearing ratio tended to be improved in the group administered with PEP013 compared to the group administered with PBS buffer (FIG. 1b).

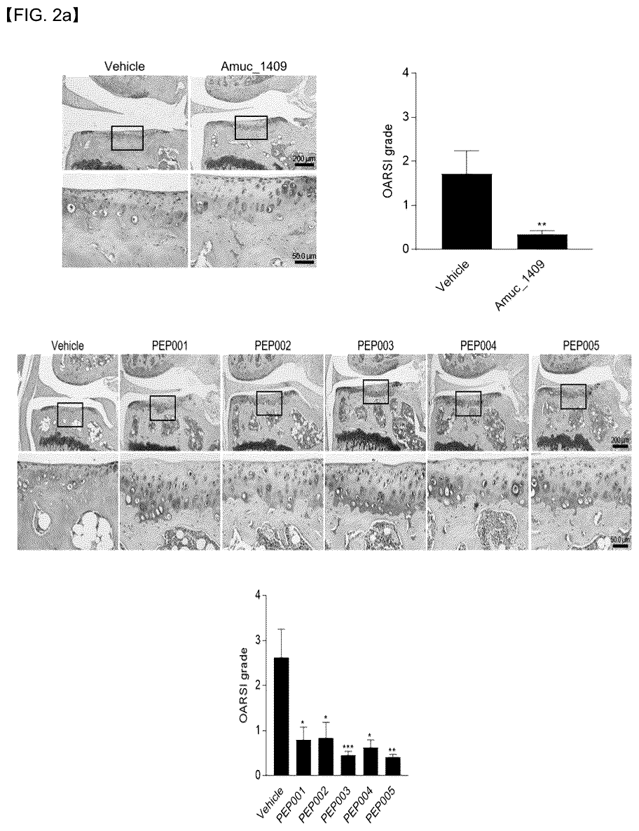

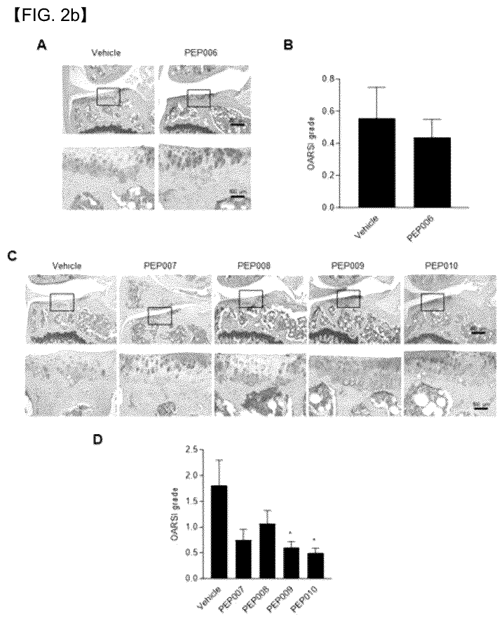

In addition, histopathological findings were examined in the mouse knee joint tissue through safranin O staining, and as a result, safranin O staining and cartilage thickness markedly decreased in the group administered with PBS buffer but only a slight loss of safranin O was observed in the cartilage zone and the overall cartilage morphology was maintained in the groups administered with Amuc_1409 and PEP001 to PEP010. When the degree of cartilage destruction was quantified quantitatively by the OARSI score, the degree of cartilage destruction significantly decreased in the groups administered with Amuc_1409 and PEP001 to PEP010 compared to the group administered with PBS buffer (p<0.05, Student's t-test) (FIGS. 2a and 2b).

### Example 1-2: Verification of Efficacy of Novel Peptides in Animal Model of Rheumatoid Arthritis

### Example 1-2-1: Experimental Method

In order to prepare a collagen induced arthritis (CIA) model, bovine type 2 collagen (CII) was dissolved in 0.1 M acetic acid solution at 2 mg/mL, then dialyzed with phosphate buffered saline, and mixed with the same volume of Complete Freund's Adjuvant (CFA, Chondrex) containing M. tuberculosis for emulsification, and the emulsified collagen solution was injected intradermally into the base of the tail of 8-week-old male DBA/1J mice (i.e., 100 μg/100 μL) to induce primary immune response (first immunization). On day 21 after the first immunization, the same CII was mixed with the same volume of CFA, and the solution was injected intradermally into the base of the tail of the mice by 100 μL (i.e., 100 μg/100 μL) to induce secondary immune response (secondary immunization). On day 28 after the first immunization, lipopolysaccharide (LPS) was intraperitoneally injected by 40 μg per animal to induce a boosting response. The experiment was terminated on day 35 after the first immunization.

From day 18 after the first immunization to the experiment end day, Amuc_1409 was dissolved in PBS buffer at a concentration of 13.4 μM and PEP001 to PEP013 at a concentration of 6.7 μM, and then injected into the mouse peritoneal cavity at 150 μL per animal once a day at the same time. Into the control group, the same volume of PBS buffer was injected intraperitoneally.

The severity of joint inflammation was evaluated at 2-day intervals from day 18 after the first immunization to the experiment end day. At this time, for arthritis evaluation, the scores for the four limbs acquired according to the scale below were summed and used. The scoring criteria for the arthritis evaluation are as follows (Table 2).

TABLE 2
CIA clinical scoring system

| Paw | |
|---|---|
| score | Clinical observations |
| 0 | Normal |
| 1 | One toe inflamed and swollen |
| 2 | Two or more toes inflamed and swollen or mild swelling of entire |
| | paw |
| 3 | Inflammation and swelling of entire paw |
| 4 | Severe inflammation and swelling of entire paw or ankylosed paw |

At the experiment end day, the thickness of the ankle joint was measured with calipers, photographs were taken for each test group, the test animals were sacrificed, the hind paws of the mice were fixed with 10% neutral buffered formalin, and the bone was decalcified with a 0.5 M EDTA solution. Next, 5 μm joint sections were prepared by embedding the mouse hind paw tissue in paraffin, and stained with hematoxylin and eosin (H&E), and the severity of joint inflammation was evaluated histopathologically. At this time, for the arthritis evaluation, the scores acquired according to the scale below were averaged and used. The scoring criteria for the arthritis evaluation are as follows.

TABLE 3
Histopathological synovitis scoring system

| Score | Synovial inflammation |
|---|---|
| 0 | No inflammation |
| 1 | Slight enlargement of synovial membrane layer or inflammatory |
| | cell influx into synovial membrane inner layer |
| 2 | Slight enlargement of synovial membrane layer and inflammatory |
| | cell influx into synovial membrane inner layer |
| 3 | Slight enlargement of synovial membrane layer and inflammatory |
| | cell influx into synovial membrane inner layer |
| | Inflammatory cell infiltration into synovial space |
| 4 | Severe infiltration of large number of inflammatory cells into |
| | synovial membrane |

### Example 1-2-2: Experimental Results

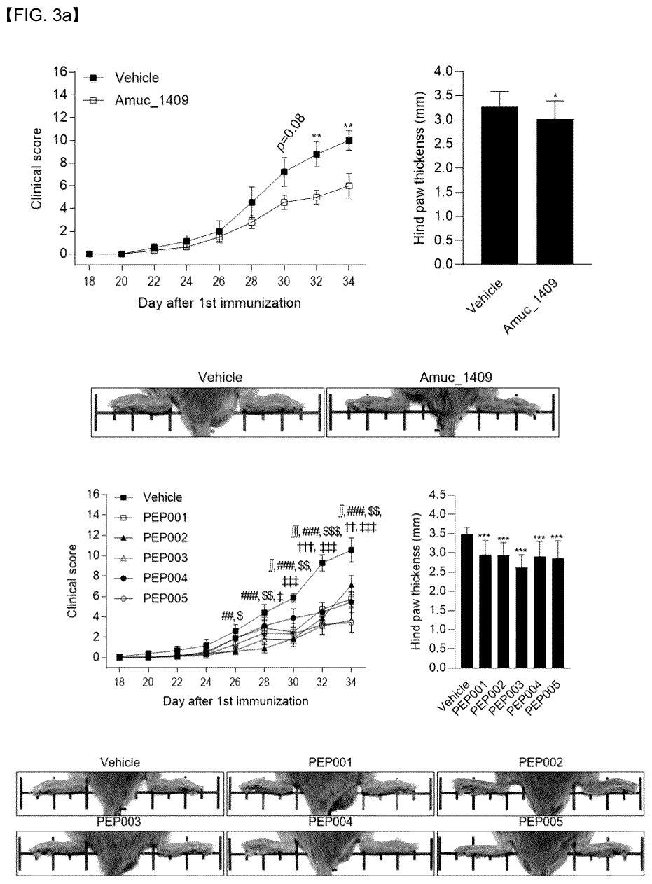

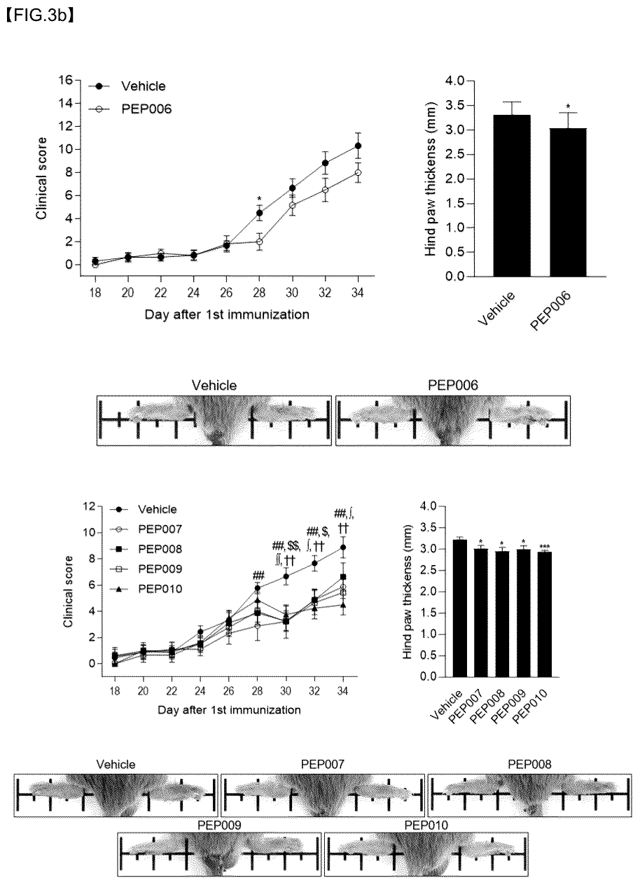

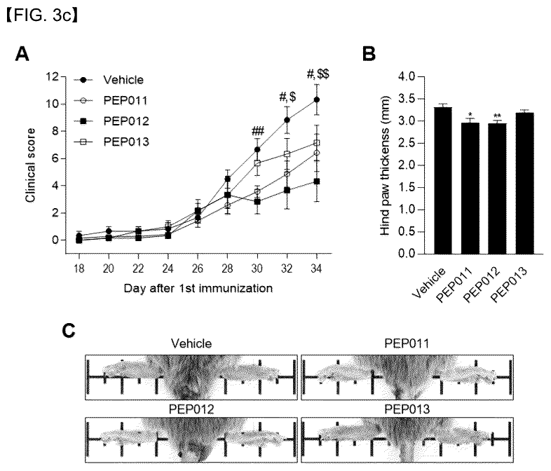

As a result of evaluating the severity of joint inflammation in the rheumatoid arthritis model, the arthritis index decreased close to a significant level in the group administered with Amuc_1409 compared to the group administered with PBS buffer on day 30 after the first CII injection, and the arthritis index significantly decreased in the group administered with Amuc_1409 compared to the group administered with PBS buffer from then until the experiment end day (p<0.01, Student's t-test). In addition, as a result of measuring the thickness of the ankle joint at the end of the experiment, the thickness of the ankle joint significantly decreased in the group administered with Amuc_1409 compared to the group administered with PBS buffer. By macrophotography as well, it was confirmed that swelling of the hind paws was reduced overall in the group administered with Amuc_1409 compared to the group administered with PBS buffer (p<0.05, Student's t-test) (FIG. 3a).

As a result of evaluating the severity of joint inflammation in the groups administered with PEP001, PEP002, PEP003, PEP004, and PEP005, compared to the group administered with PBS buffer, the arthritis index significantly decreased from day 30 after the first CII injection to the experiment end day in the group administered with PEP001, the arthritis index significantly decreased from day 26 after the first CII injection to the experiment end day in the groups administered with PEP002 and PEP003, the arthritis index significantly decreased from day 32 after the first CII injection to the experiment end day in the group administered with PEP004, and the arthritis index significantly decreased from day 28 after the first CII injection to the experiment end day in the group administered with PEP005. In addition, as a result of measuring the thickness of the ankle joint at the end of the experiment, the thickness of the ankle joint significantly decreased in the groups administered with PEP001, PEP002, PEP003, PEP004, and PEP005 compared to the group administered with PBS buffer. By macrophotography as well, it was confirmed that swelling of the hind paws was reduced overall in the groups administered with PEP001, PEP002, PEP003, PEP004, and PEP005 compared to the group administered with PBS buffer (p<0.05, Student's t-test) (FIG. 3a).

As a result of evaluating the severity of joint inflammation in the groups administered with the novel peptides PEP006, PEP007, PEP008, PEP009, and PEP010, the arthritis index significantly decreased on day 28 after the first injection of CII and tended to decrease from day 30 to the experiment end day in the group administered with PEP006 compared to the group administered with PBS buffer. In addition, compared to the group administered with PBS buffer, the arthritis index significantly decreased from day 28 after the first CII injection to the experiment end day in the group administered with PEP007, the arthritis index significantly decreased from day 30 to day 32 after the first CII in the group administered with PEP008, and the arthritis index significantly decreased from day 30 after the first CII injection to the experiment end day in the groups administered with PEP009 and PEP010. In addition, as a result of measuring the thickness of the ankle joint at the end of the experiment, the thickness of the ankle joint significantly decreased in the groups administered with PEP006, PEP007, PEP008, PEP009, and PEP010 compared to the group administered with PBS buffer. By macrophotography as well, it was confirmed that swelling of the hind paws was remarkably improved in the groups administered with PEP006, PEP007, PEP008, PEP009, and PEP010 compared to the group administered with PBS buffer (p<0.05, Student's t-test) (FIG. 3b).

As a result of evaluating the severity of joint inflammation in the groups administered with PEP011, PEP012, and PEP013, compared to the group administered with PBS buffer, the arthritis index significantly decreased from day 30 after the first CII injection to the experiment end day in the group administered with PEP011, the arthritis index significantly decreased from day 32 after the first CII injection to the experiment end day in the group administered with PEP012, and the arthritis index tended to decrease in the group administered with PEP013. In addition, as a result of measuring the thickness of the ankle joint at the end of the experiment, the thickness of the ankle joint significantly decreased in the groups administered with PEP011 and PEP012 and tended to decrease in the group administered with PEP013 compared to the group administered with PBS buffer. By macrophotography as well, it was confirmed that swelling of the hind paws was relieved overall in the groups administered with PEP011, PEP012, and PEP013 compared to the group administered with PBS buffer (p<0.05, Student's t-test) (FIG. 3c).

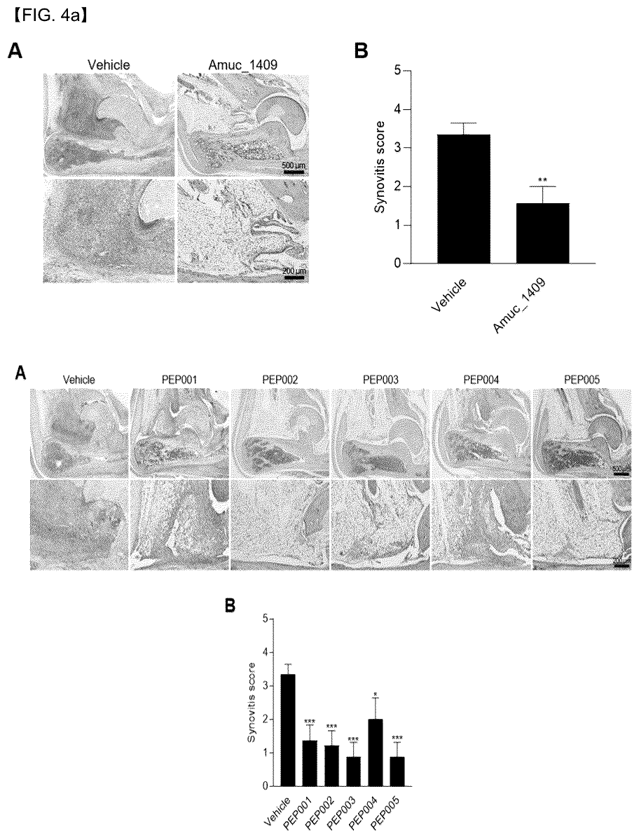

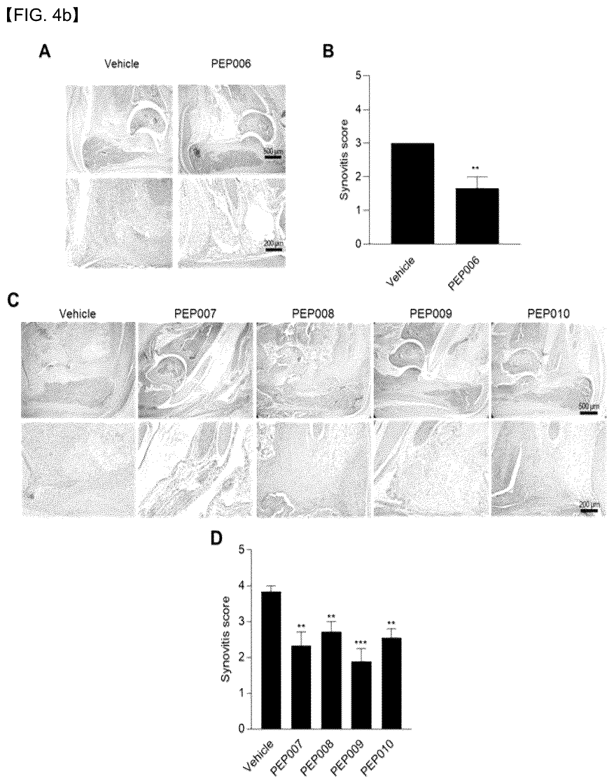

In addition, as a result of examining histopathological findings in the mouse hind paw tissue through H&E staining, moderate inflammatory cell infiltration, clear synovial proliferation, and pannus formation in the joint cavity were observed in the group administered with PBS buffer, but the histopathological changes observed in the group administered with PBS buffer were significantly diminished in the groups administered with Amuc_1409 and PEP001 to PEP013. The quantitative histopathological arthritis index also significantly decreased or tended to decrease in the groups administered with Amuc_1409 and PEP001 to PEP012 compared to the group administered with PBS buffer (p<0.05, Student's t-test) (FIG. 4a, FIG. 4b and FIG. 4c).

Through this, it has been confirmed that the peptides of the present invention have an effect of preventing and treating arthritis.

### Example 2: Verification of Efficacy of Novel Peptides in Animal Model of Metabolic Disease

### Example 2-1: Experimental Method

An animal model of metabolic disease accompanied by obesity/diabetes and chronic inflammation was induced by feeding a high-fat diet.

The experiment in the groups administered with Amuc_1409 or PEP001 to PEP005 was performed using 8-week-old SPF male C57BL/6J mice, and the metabolic disease accompanied by obesity/diabetes and chronic inflammation was induced by ad libitum feeding of a high-fat diet for 45 weeks or 8 weeks. Specifically, mice fed a high-fat diet for 45 weeks were fed a high-fat diet for an additional 3 weeks, and Amuc_1409 was dissolved in PBS buffer at a concentration of 2.4 μM and then orally administered to the mice by 150 μL per animal at the same time once a day until the end of the experiment, or mice fed a high-fat diet for 8 weeks were fed a high-fat diet for an additional 3 weeks, and PEP001, PEP002, PEP003, PEP004, or PEP005 was dissolved in PBS buffer at a concentration of 2.4 μM and then orally administered to the mice by 150 μL per animal at the same time once a day until the end of the experiment. To the control group, PBS buffer was orally administered in the same manner.

For the experiment in the groups administered with PEP006 to PEP013, 8-week-old male C57BL/6J mice were used, and the metabolic disease accompanied by obesity/diabetes and chronic inflammation was induced by ad libitum feeding of a high-fat diet for 32, 50, or 53 weeks. Mice fed a high-fat diet for 32 weeks were fed a high-fat diet for an additional 3 weeks, and a novel tetrapeptide PEP006 was dissolved in PBS buffer at a concentration of 0.15 μg/150 μL (PEP006) and then orally administered to the mice by 150 μL per animal at the same time until the end of the test, mice fed a high-fat diet for 50 weeks were fed a high-fat diet for an additional 3 weeks, and a novel tetrapeptide PEP007, PEP008, PEP009, or PEP010 was dissolved in PBS buffer at a concentration of 0.2 μg/150 μL (PEP007 to PEP013) and then orally administered to the mice by 150 μL per animal at the same time until the end of the test, and mice fed a high-fat diet for 53 weeks were fed a high-fat diet for an additional 3 weeks, and a novel tetrapeptide PEP011, PEP012, or PEP013 was dissolved in PBS buffer at a concentration of 0.2 μg/150 μL (PEP007 to PEP013) and then orally administered to the mice by 150 μL per animal at the same time until the end of the test. To the control group, PBS buffer was orally administered in the same manner.

For all animals, general symptoms were observed immediately before administration at the start of administration, and the body weight was measured until the end of the test after group separation.

Insulin (Humalog, 1 unit/kg) was intraperitoneally injected into mice fasted for 4 hours at 2 weeks after the start of test substance administration (2 weeks in groups administered with Amuc_1409 and PEP001 to PEP005 and 3 weeks in groups administered with PEP006 to PEP013), and then blood glucose at 30 minutes, 60 minutes, 90 minutes, and 120 minutes was measured from a small amount of blood obtained through a wound at the tip of the tail using ACCU-CHECK (Roche), thereby performing an insulin tolerance test (ITT).

At the end of the test, fasting blood glucose was measured from a small amount of blood obtained through a wound at the tip of the tail of the mouse fasted for 16 hours using ACCU-CHECK, and blood was collected from the orbital venous plexus using a heparin-treated capillary tube. The collected blood was immediately centrifuged at 10,000 rpm (4° C.) for 10 minutes to separate plasma, and then subjected to analysis of total cholesterol (TC) and low-density lipoprotein cholesterol (LDL-C), which are blood abnormal lipid indicators, using an automated blood biochemical analyzer.

### Example 2-2: Experimental Results

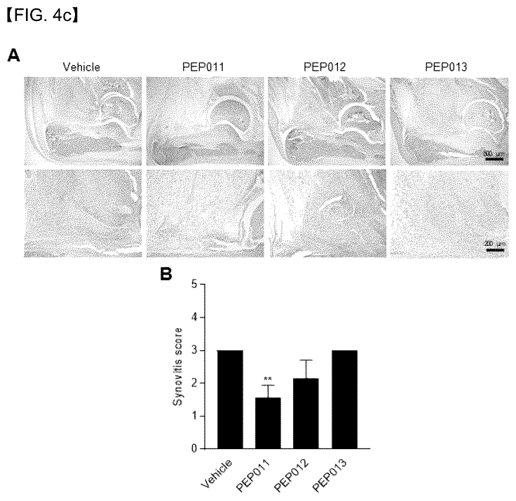

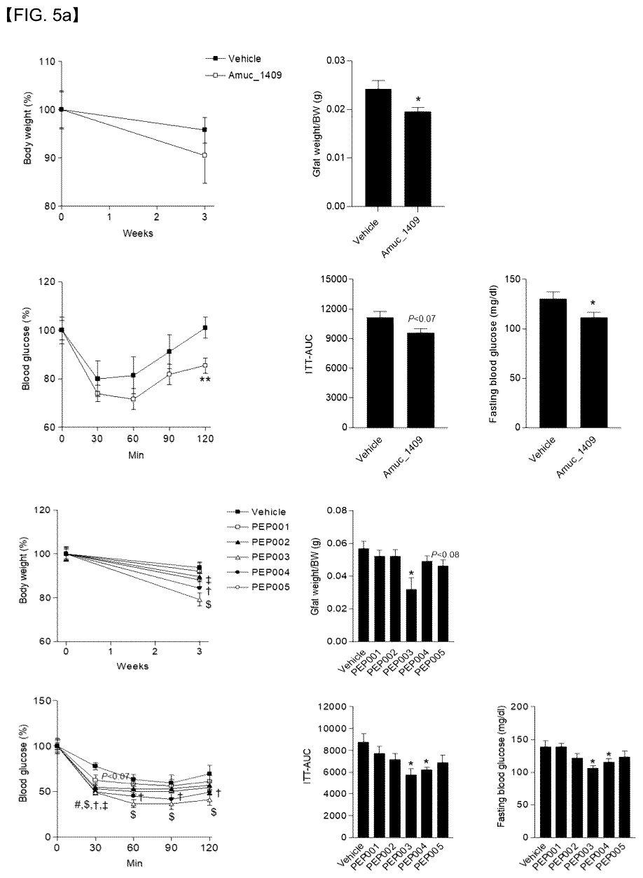

In an animal model of metabolic disease accompanied by obesity/diabetes and chronic inflammation, the body weight decreased from the initial body weight in the group administered with Amuc_1409 compared to the group administered with PBS buffer, and abdominal fat significantly decreased in the group administered with Amuc_1409, showing an anti-obesity effect (p<0.05, Student's t-test) (FIG. 5a).

In the insulin resistance experiment, the blood glucose decreased from 30 minutes to 120 minutes after insulin administration in the group administered with Amuc_1409 compared to the group administered with PBS buffer. In the group administered with Amuc_1409, the decreased blood glucose was significantly maintained until 120 minutes, showing the effect of improving insulin resistance by a high-fat diet. As a result of converting the graph of the insulin resistance experiment into AUC (area under the curve), it was confirmed that the AUC value decreased close to a significant level in the group administered with Amuc_1409 compared to the group administered with PBS buffer. In addition, when fasting blood glucose was measured after fasting for 16 hours at the end of the test, it was confirmed that the fasting blood glucose significantly decreased in the group administered with Amuc 1409 (p<0.05, Student's t-test) (FIG. 5a).

In an animal model of metabolic disease accompanied by obesity/diabetes and chronic inflammation, the body weight decreased from the initial body weight in all of the groups administered with PEP001, PEP002, PEP003, PEP004, and PEP005 compared to the group administered with PBS buffer, and the body weight significantly decreased in the groups administered with PEP003, PEP004, and PEP005. Abdominal fat decreased in all of the groups administered with PEP001, PEP002, PEP003, PEP004, and PEP005 compared to the group administered with PBS buffer, showing an anti-obesity effect (p<0.05, Student's t-test) (FIG. 5a).

In the insulin resistance experiment, the blood glucose decreased at 30 minutes after insulin administration in all of the groups administered with PEP001, PEP002, PEP003, PEP004, and PEP005 compared to the group administered with PBS buffer, and the decreased blood glucose was significantly maintained until 120 minutes in the groups administered with PEP003 and PEP004, showing the effect of improving insulin resistance by a high-fat diet. As a result of converting the graph of the insulin resistance experiment into AUC (area under the curve), it was confirmed that the AUC value decreased in all of the groups administered with PEP001, PEP002, PEP003, PEP004, and PEP005 compared to the group administered with PBS buffer. In addition, when fasting blood glucose was measured after fasting for 16 hours at the end of the test, it was confirmed that the fasting blood glucose decreased in the groups administered with PEP002, PEP003, PEP004, and PEP005 (p<0.05, Student's t-test) (FIG. 5a).

In the insulin resistance experiment, the blood glucose decreased to a significant level from 30 minutes to 120 minutes after insulin administration in the groups administered with novel tetrapeptides PEP006, PEP007, PEP008, PEP009, and PEP010 compared to the group administered with PBS buffer. As a result of converting the graph of the insulin resistance experiment into AUC (area under the curve), compared to the group administered with PBS buffer, the AUC value significantly decreased in the group administered with PEP008, the AUC value decreased close to a significant level in the groups administered with PEP006, PEP007, PEP009, and PEP010, and the efficacy of improving insulin resistance by a high-fat diet was confirmed (p<0.05, Student's t-test) (FIG. 5b).

In the insulin resistance experiment, when the novel tripeptides PEP011, PEP012, and PEP013 were administered as well, the blood glucose decreased close to a significant level from 30 minutes to 120 minutes after insulin administration compared to the group administered with PBS buffer. As a result of converting the graph of the insulin resistance experiment into AUC (area under the curve), compared to the group administered with PBS buffer, the AUC value decreased close to a significant level in the groups administered with PEP011, PEP012, and PEP013, and efficacy of improving insulin resistance by a high-fat diet was confirmed (p<0.05, Student's t-test) (FIG. 5b).

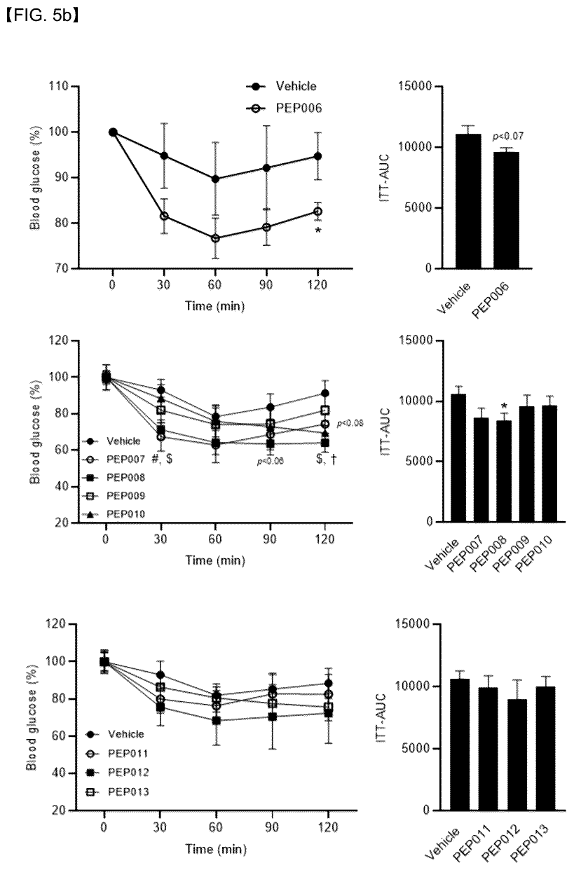

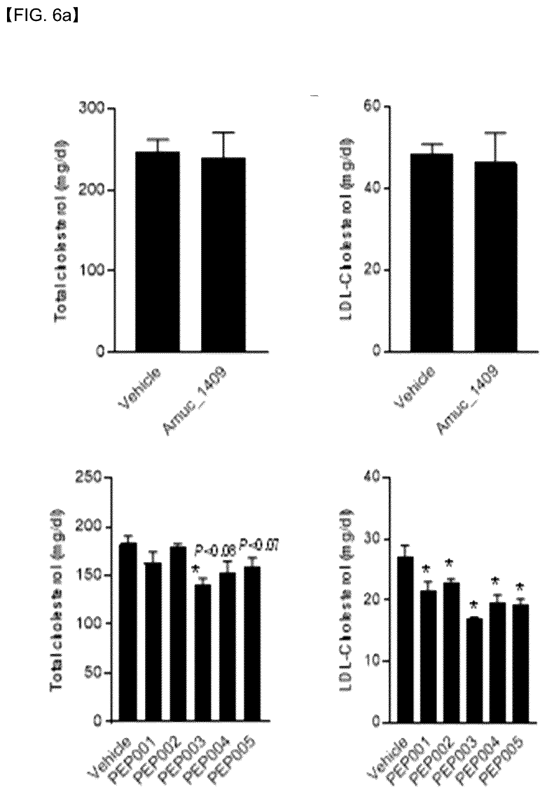

TC and LDL-C levels, which are blood lipid indicators, decreased in the groups administered with PEP001, PEP002, PEP003, PEP004, and PEP005 compared to the group administered with PBS buffer. TC significantly decreased in the group administered with PEP003 and tended to decrease in the groups administered with PEP004 and PEP005. In addition, LDL-C significantly decreased in all of the groups administered with PEP001, PEP002, PEP003, PEP004, and PEP005 compared to the group administered with PBS buffer, showing the effect of improving dyslipidemia (p<0.05, Student's t-test) (FIG. 6a). In the groups administered with PEP007 to PEP013 as well, the TC level tended to decrease and the LDL-C level tended to drop, and efficacy of improving dyslipidemia was confirmed (FIGS. 6a and 6b).

### Example 3: Verification of Efficacy of Novel Peptides in Animal Model of Hepatitis

### Example 3-1: Verification of Efficacy of Novel Peptide in Animal Model of Acute Hepatitis

### Example 3-1-1: Experimental Method

An acute hepatitis model was induced by dissolving ConA (concanavalin A) in PBS buffer (phosphate buffered saline) at 1.5 mg/mL and injecting the solution into 8-week-old specific pathogen free (SPF) male C57BL/6J mice through the tail vein in proportion to the mouse body weight based on 100 μL/10 g.

Amuc_1409 or PEP001, PEP002, PEP003, PEP004, or PEP005 was dissolved in PBS buffer at a concentration of 6.7 μM from 3 days before ConA administration, and PEP007, PEP008, PEP009, or PEP010 was dissolved in PBS buffer at a concentration of 0.2 μg/150 μL from 3 days before ConA administration, and Amuc_1409 was injected into the mouse peritoneal cavity once a day at the same time by 210 μL and PEP001 to PEP010 by 150 μL, and intraperitoneally injected additionally one time 30 minutes before ConA injection, and the experiment was terminated 9 hours after ConA intravenous injection. Into the control group, PBS buffer was intravenously injected in the same manner.

At the end of the experiment 9 hours after ConA intravenous injection, blood was collected from the orbital venous plexus using a heparin-treated capillary tube, and the collected blood was immediately centrifuged at 10,000 rpm (4° C.) for 10 minutes to separate plasma and then subjected to the analysis of alanine aminotransferase (ALT) and aspartate aminotransferase (AST), which are indicators of liver damage, using an automatic blood biochemical analyzer (Beckman Coulter AU480 automatic analyzer, Beckman Coulter). The liver was harvested, a part of the liver was fixed in 10% NBF at room temperature for 24 hours or more and then embedded in paraffin to prepare a 5 μm-thick section, and the section was stained with hematoxylin and eosin (H&E).

### Example 3-1-2: Experimental Results

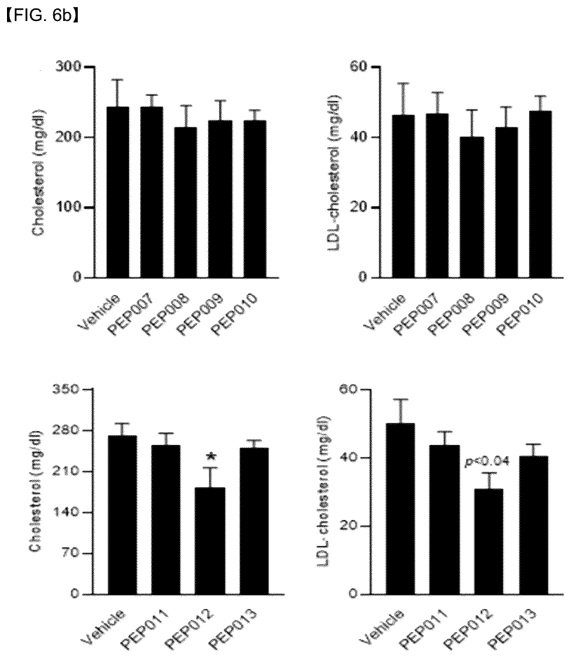

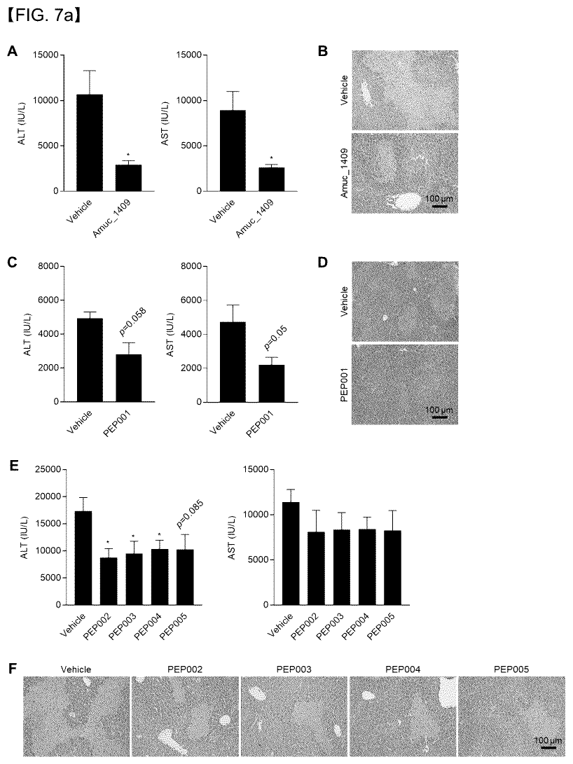

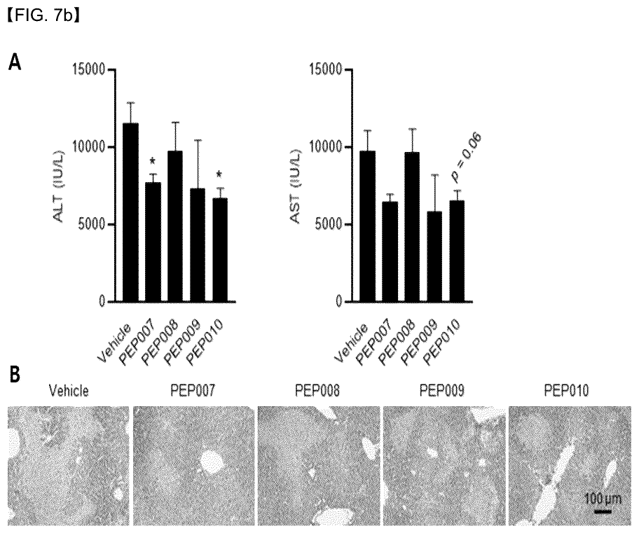

As a result of analyzing ALT and AST, which are indicators of liver damage in an acute hepatitis model, it was confirmed that ALT significantly decreased in the groups administered with Amuc_1409, PEP002, PEP003, and PEP004 and decreased close to a significant level in the groups administered with PEP001 and PEP005 compared to the group administered with PBS buffer. Meanwhile, AST significantly decreased in the group administered with Amuc 1409, decreased close to a significant level in the group administered with PEP001, and tended to decrease in the groups administered with PEP002, PEP003, PEP004, and PEP005 compared to the group administered with PBS buffer (p<0.05, Student's t-test) (FIG. 7a). In addition, similar tendency was confirmed in the groups administered with PEP007 to PEP010 as well. Specifically, ALT significantly decreased in the groups administered with the novel tetrapeptides PEP007 and PEP010 and tended to decrease in the groups administered with PEP008 and PEP009 compared to the group administered with PBS buffer. AST decreased close to a significant level in the group administered with PEP010 and tended to decrease by administration of PEP007 and PEP009 compared to the group administered with PBS buffer (p<0.05, Student's t-test) (FIG. 7b).

In addition, as a result of examining histopathological findings in the liver tissue through H&E staining, inflammatory cells infiltration into the liver and necrosis in the liver parenchyma following the occurrence of ConA-induced acute hepatitis were markedly reduced in the groups administered with Amuc_1409 and PEP001 to PEP010 compared to the group administered with PBS buffer (FIGS. 7a and 7b).

### Example 3-2: Verification of Efficacy of Novel Peptides in Animal Model of Non-Alcoholic Steatohepatitis

### Example 3-2-1: Experimental Method

A non-alcoholic hepatitis model was induced by feeding a high-fat diet to 8-week-old SPF male C57BL/6J mice.

The experiment for groups administered with Amuc_1409 and PEP001 to PEP005 was performed as follows. Mice fed a high-fat diet for 45 weeks were fed a high-fat diet for an additional 3 weeks, and Amuc_1409 was dissolved in PBS buffer at a concentration of 2.4 μM and then orally administered to the mice by 150 μL per animal at the same time once a day until the end of the experiment, or mice fed a high-fat diet for 8 weeks were fed a high-fat diet for an additional 3 weeks, and PEP001, PEP002, PEP003, PEP004, or PEP005 was dissolved in PBS buffer at a concentration of 2.4 μM and then orally administered to the mice by 150 μL per animal at the same time once a day until the end of the experiment. To the control group, PBS buffer was orally administered in the same manner.

The experiment for groups administered with PEP006 to PEP013 was performed as follows. The non-alcoholic hepatitis model was induced by ad libitum feeding of a high-fat diet for 32, 50, or 53 weeks. Mice fed a high-fat diet for 50 weeks were fed a high-fat diet for an additional 3 weeks, and a novel tetrapeptide PEP006, PEP007, PEP008, PEP009, or PEP010 was dissolved in PBS buffer at a concentration of 0.2 μg/150 μL and then orally administered to the mice by 150 μL per animal at the same time until the end of the test, and mice fed a high-fat diet for 53 weeks were fed a high-fat diet for an additional 3 weeks, and a novel tetrapeptide PEP011, PEP012, or PEP013 was dissolved in PBS buffer at a concentration of 0.2 μg/150 μL and then orally administered to the mice by 150 μL per animal at the same time until the end of the test. To the control group, PBS buffer was orally administered in the same manner.

At the end of the experiment, a part of the liver harvested was homogenized with Trizol to extract RNA. Reverse transcriptase was added to the obtained RNA and reacted to synthesize cDNA, and SYBR Green and primers were added to perform quantitative real-time PCR (RT-qPCR), and then the expression levels of genes such as Tnfα, Il-1β, Il-10, and Il-1rn were quantified.

### Example 3-2-2: Experimental Results

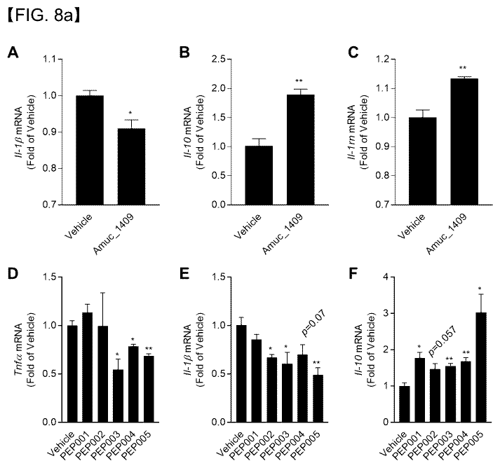

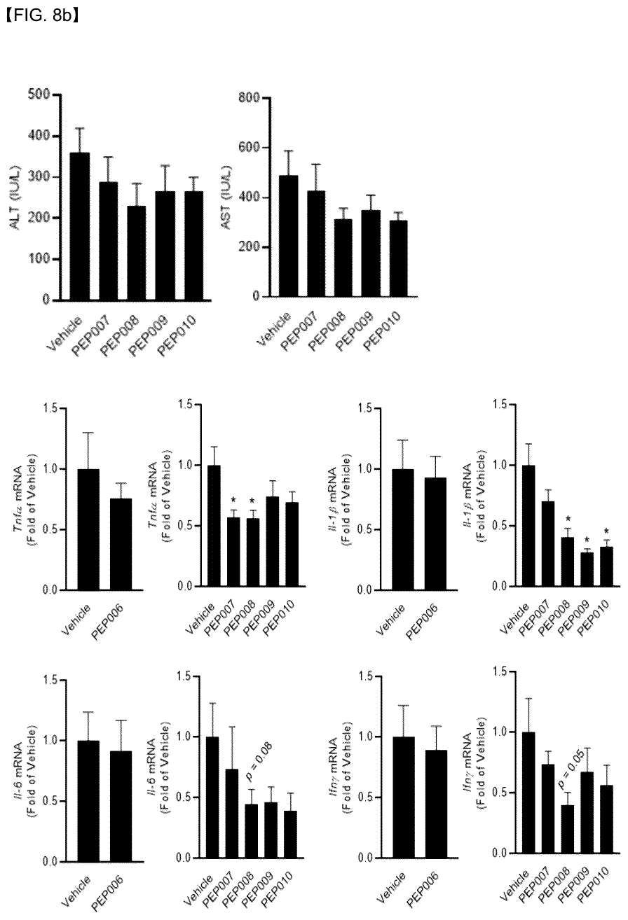

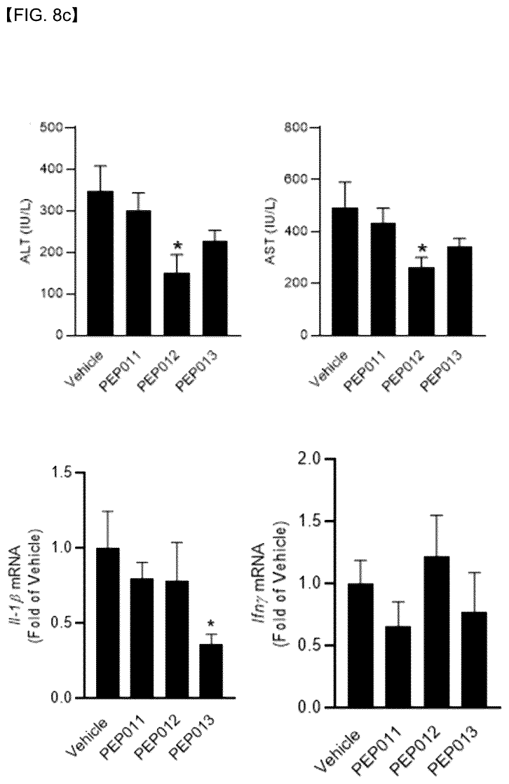

As a result of examining gene expression in the liver tissue of the non-alcoholic hepatitis model, inflammatory markers decreased and anti-inflammatory markers increased by peptide administration over all. Specifically, among the inflammatory markers that increase in high-fat diet-induced non-alcoholic hepatitis, Tnfα significantly decreased in the groups administered with PEP003, PEP004, PEP005, PEP007, and PEP008 and tended to decrease in the groups administered with PEP006, PEP009, and PEP010 compared to the group administered with PBS buffer. Il-1p decreased close to a significant level in the groups administered with PEP001 and PEP004, significantly decreased in the groups administered with Amuc_1409, PEP002, PEP003, PEP005, PEP008, PEP009, PEP010, and PEP013, and tended to decrease in the groups administered with PEP011 and PEP012. Il-6 tended to decrease in the groups administered with PEP007, PEP008, PEP009, and PEP010. Ifnγ decreased close to a significant level in the group administered with PEP008, and tended to decrease in the groups administered with PEP007, PEP009, PEP010, PEP011, and PEP013 (FIG. 8a, FIG. 8b, and FIG. 8c).

Meanwhile, 11-10, which is an anti-inflammatory index, significantly increased in the groups administered with Amuc_1409, PEP001, PEP003, PEP004, and PEP005 and tended to increase close to a significant level in the group administered with PEP002 compared to the group administered with PBS buffer. Il-1rn, which is an additional anti-inflammatory index, significantly increased in the group administered with Amuc_1409 compared to the group administered with PBS buffer (FIG. 8a).

Through this, it has been confirmed that the peptides of the present invention have an effect of preventing and treating hepatitis.

### Example 4: Verification of Efficacy of Novel Peptides in Animal Model of Inflammatory Bowel Disease

### Example 4-1: Experimental Method

In order to confirm the efficacy of the peptides of the present invention for preventing and treating inflammatory bowel disease, an experiment was conducted using an animal model of enteritis.

As the enteritis model, enteritis was induced by dissolving DSS in drinking water at a concentration of 1.2% and then administered to 10-week-old specific pathogen free (SPF) male C57BL/6J mice through the drinking water for 6 days, and then the drinking water was replaced with water to give a recovery period.

During the recovery period, PEP002, PEP003, PEP004, or PEP005 was dissolved in PBS buffer at a concentration of 4.3 μM and then orally administered to the mice by 150 μL per animal once a day at the same time, and the same volume of PBS buffer was orally administered to the control group.

During the recovery period, oral administration was performed 6 times for 6 days to examine the rate of increase in body weight due to recovery from enteritis. In addition, in order to examine the length of the colon, the degree of inflammation in the colon, and the degree of recovery of the colon tissue, the test was terminated after oral administration 3 times for 3 days.

At the end of the experiment, the length of the colon was measured, a part of the distal colon was rapidly cooled in liquid nitrogen, and RNA was extracted using Trizol. Reverse transcriptase was added to the obtained RNA and reacted to synthesize cDNA, and SYBR Green and primers were added to perform quantitative real-time PCR (RT-qPCR), and then the level of Il-6 gene expression was quantified. In addition, a part of the distal colon was fixed in 10% NBF at room temperature for 24 hours or more, and then embedded in paraffin to prepare a 5 μm-thick section, and the section was stained with hematoxylin and eosin (H&E). The H&E-stained tissue sections were observed under an optical microscope and the histopathological degree of enteritis was evaluated using a histologic colitis scoring system (Table 4).

TABLE 4
Histologic colitis scoring system

| Feature score | Score | Description |
|---|---|---|
| Inflammation severity | 0 | None |
| | 1 | Mild |
| | 2 | Moderate |
| | 3 | Severe |
| Inflammation extent | 0 | None |
| | 1 | Mucosa |
| | 2 | Submucosa |
| | 3 | Transmural |
| Crypt damage | 0 | None |
| | 1 | Basal ⅓ damage |
| | 2 | Basal ⅔ damage |
| | 3 | Crypt lost; surface epithelium present |
| | 4 | Crypt and surface epithelium lost |
| Percent involvement | 0 | 0% |
| | 1 | 1-25% |
| | 2 | 26-50% |
| | 3 | 51-75% |
| | 4 | 76-100% |

### Example 4-2: Experimental Results

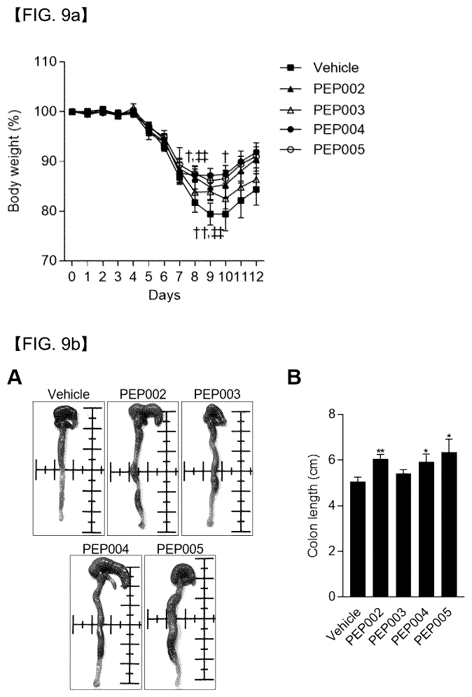

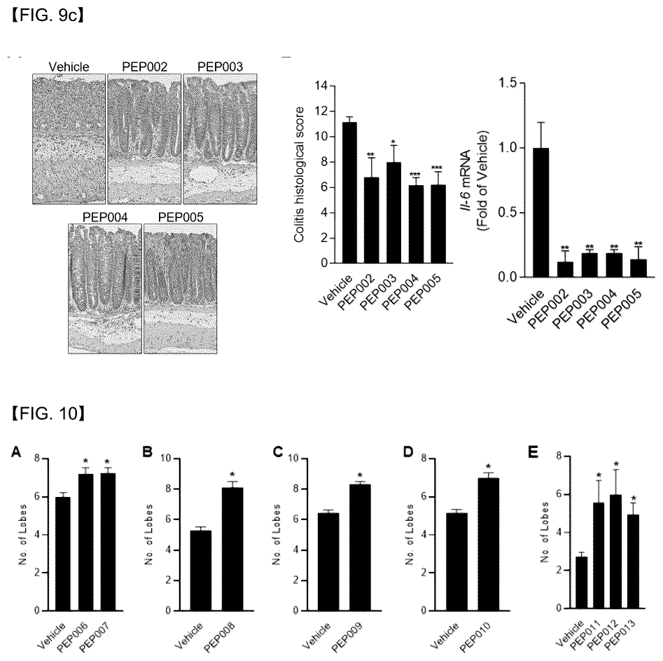

As a result of the analysis, as a result of analyzing the weight gain rate in the enteritis model, the body weight significantly increased in the groups administered with PEP004 and PEP005 and tended to increase in the groups administered with PEP002 and PEP003 compared to the group administered with PBS buffer (p<0.05, Student's t-test) (FIG. 9a).

In addition, as a result of measuring the length of the colon, the length of the colon significantly increased in the groups administered with PEP002, PEP004, and PEP005 and tended to increase in the group administered with PEP003 compared to the group administered with PBS buffer (p<0.05, Student's t-test) (FIG. 9b).

As a result of histological examination through H&E staining, the degree or extent of intestinal inflammation decreased and the degree of crypt damage was observed to significantly decrease in the groups administered with PEP002, PEP003, PEP004, and PEP005 compared to the group administered with PBS buffer (p<0.05, Student's t-test) (FIG. 9c).

In addition, Il-6 gene expression significantly decreased in the groups administered with PEP002, PEP003, PEP004, and PEP005 compared to the group administered with PBS buffer (p<0.05, Student's t-test) (FIG. 9c).

Through this, it has been confirmed that the peptides of the present invention have an effect of preventing and treating inflammatory bowel disease.

### Example 4-3. Verification of Efficacy of Novel Peptides in Small Intestine Organoid Model

### Example 4-3-1. Experiment Method

In order to establish a small intestine organoid model, the small intestine of 15-week-old male C57BL/6J mice was cut longitudinally from the proximal end to the distal end, and cut transversely into about 5 mm long pieces. The small intestine tissue thus obtained was washed with refrigerated PBS containing 1% penicillin. Thereafter, the washed small intestine tissue was treated with dissociation buffer and filtered through a cell strainer to isolate crypts. The isolated crypt was cultured in Matrigel and Organoid Growth Medium (Intesticult™) mixed at a ratio of 1:1, Matrigel was solidified at 37° C. for 20 minutes in a 12 well plate, and then medium (500 μL/well) was added. The medium was replaced after 4 days, and subculture was performed on day 7.

For novel peptide screening, organoids 3D cultured in medium and Matrigel support were dispensed into 3 wells per group using a 12-well plate and 50 organoids per well. On days 0, 2, and 4 of culture, the organoids were treated with novel tetrapeptides PEP006, PEP007, PEP008, PEP009, and PEP010 and novel tripeptides PEP011, PEP012, and PEP013 at a concentration of 16 nM as described above, and the number of lobes of sprouted organoids on day 5 of culture was examined and analyzed for comparison. The control group was treated with PBS buffer in the same manner.

As a result of the analysis, the number of lobes of sprouted organoids significantly increased in the groups treated with the novel peptides PEP006, PEP007, PEP008, PEP009, PEP010, PEP011, PEP012, and PEP013 compared to the group administered with PBS buffer (FIG. 10).

Through this, it has been confirmed that the peptides of the present invention have an effect of regenerating intestinal tissue as well and can be utilized for prevention and treatment of various intestinal diseases including inflammatory bowel disease.

### Example 5: Verification of Efficacy of Novel Peptides in Animal Model of Gastritis/Gastric Ulcer

### Example 5-1: Experimental Method

For a gastritis/gastric ulcer model, 7-week-old male C57BL/6J mice were used, and from 3 days before induction, the peptides of the present invention were dissolved in PBS buffer and then orally administered to the mice by 150 μL per animal at the same time once a day. Amuc_1409 or PEP001, PEP002, PEP003, PEP004, or PEP005 was dissolved in PBS buffer at a concentration of 6.7 μM, and novel tetrapeptides PEP006, PEP007, PEP008, PEP009, and PEP010 and novel tripeptides PEP011, PEP012, and PEP013 were each dissolved in PBS buffer at a concentration of 0.15 μg/150 μL (PEP006) or 0.2 μg/150 μL (PEP007, PEP008, PEP009, PEP010, PEP011, PEP012, and PEP013).

For the induction of acute gastritis/gastric ulcer, 0.3 M HCl/60% ethanol was orally administered to the mice by 0.2 mL for each, the tissue was harvested after 1 hour, photographs of the inner surface of the stomach were taken, and the degree of damage to the stomach was compared.

Measurements of gastritis/gastric ulcer indexes were converted into scores according to the degree of mucosal damage. A score of 0 was given if there was no damage, a score of 1 was given to an insignificant circle-shaped hemorrhagic corrosion site of less than 5 mm, a score of 2 was given to a hemorrhagic corrosion site with a length more than 5 mm, and a score of 3 was given to one or more hemorrhagic corrosion sites corresponding to the score of 2, a score of 4 was given to a hemorrhagic corrosion of more than 5 mm in length and less than 2 mm in width, a score of 5 was given to 2 to 3 hemorrhagic corrosions corresponding to the score of 4, a score of 6 was given to 4 to 5 hemorrhagic corrosions corresponding to the score of 4, a score of 7 was given to 6 or more hemorrhagic corrosions corresponding to the score of 4, and a score of 8 was given if there was hemorrhagic corrosion throughout the gastric mucosa.

In order to confirm additional anti-inflammatory activity of the administered peptides, mRNA expression of inflammation-related genes, Tnfα, Il-1β, iNos, Cox2, and Il-10, in the gastric mucosal tissue was examined.

### Example 5-2: Experimental Results

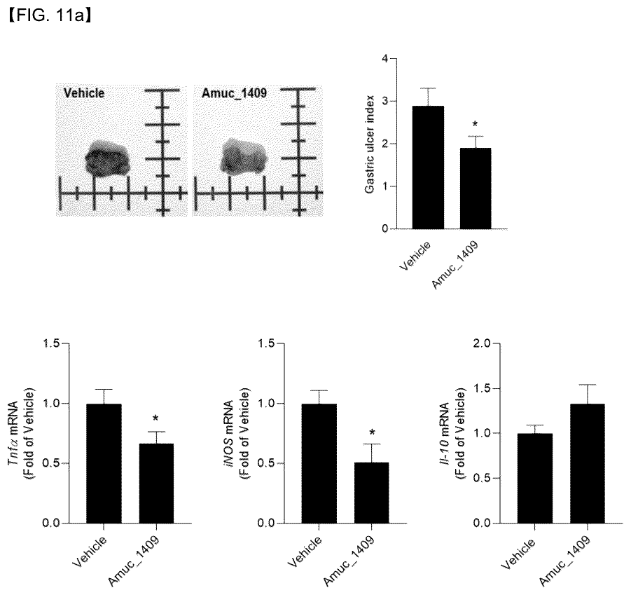

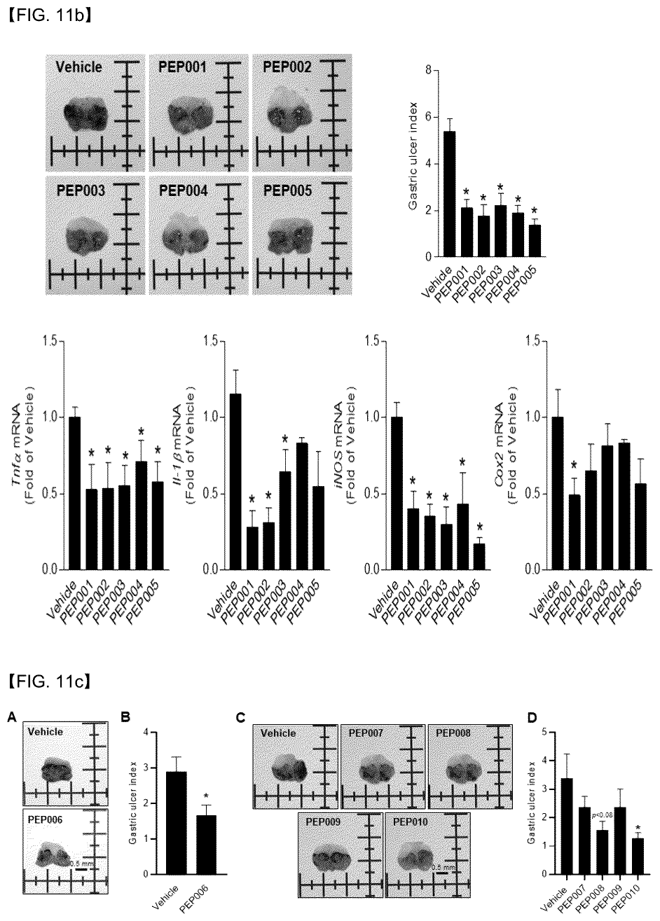

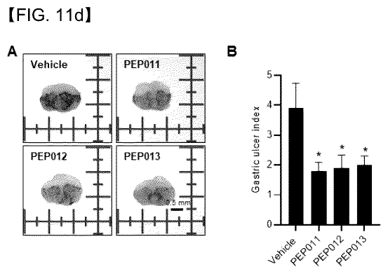

When the degree of gastric mucosal damage was scored in the gastritis/gastric ulcer model prepared in Example 5-1, the degree of gastric mucosal damage significantly decreased in the group administered with Amuc_1409 compared to the group administered with PBS buffer (p<0.05, Student's t-test) (FIG. 11a).

In addition, as a result of analyzing the expression of gastric mucosal inflammation-related genes through RT-qPCR, the mRNA expression of Tnfα and iNos significantly decreased and mRNA expression of Il-10, which is an anti-inflammatory cytokine, increased in the group administered with Amuc_1409 compared to the group administered with PBS buffer (p<0.05, Student's t-test) (FIG. 11a).

When the degree of gastric mucosal damage was scored in the gastritis/gastric ulcer model, the degree of gastric mucosal damage significantly decreased in the groups administered with PEP001, PEP002, PEP003, PEP004, and PEP005 compared to the group administered with PBS buffer (p<0.05, Student's t-test) (FIG. 11b).

In addition, as a result of analyzing the expression of gastric mucosal inflammation-related genes through RT-qPCR, the expression of Tnfα, Il-1p, iNos, and Cox2 significantly decreased in the groups administered with PEP001, PEP002, PEP003, PEP004, and PEP005 compared to the group administered with PBS buffer (p<0.05, Student's t-test) (FIG. 11b).

Similarly, the degree of gastric mucosal damage significantly decreased in the groups administered with the novel tetrapeptides PEP006 and PEP010 and the novel tripeptides PEP011, PEP012, and PEP013 compared to the group administered with PBS buffer, and the degree of gastric mucosal damage decreased to a significant level in the groups administered with PEP007, PEP008, and PEP009 compared to the group administered with PBS buffer (p<0.05, Student's t-test) (FIGS. 11c and 11d).

Through this, it has been confirmed that the peptides of the present invention have an effect of preventing and treating gastritis and gastric ulcer.

### Example 6: Verification of Efficacy of Novel Peptides in Animal Model of Dermatitis

### Example 6-1: Experimental Method

As a dermatitis model, one day before sample application, both ears of 8-week-old male C57BL/6J mice were shaved, 2.5 μg of TPA (12-otetrade-canoyl-phorbol-13-acetate) dissolved in 25 μL acetone was applied to the right ear of the mouse to induce skin swelling and inflammation, and the left ear was coated with 25 μL of acetone, which is a TPA solvent, and used as a negative control.

From 3 days before TPA application, Amuc_1409 or PEP001 to PEP013 were each dissolved in PBS buffer at a concentration of 6.7 μM, and then injected into the abdominal cavity of mice by 150 μL per animal once a day at the same time. On day 3 (0 days) after administration of the peptide, the peptide was injected intraperitoneally one time 30 minutes before TPA application, and the drug was additionally injected intraperitoneally 3 hours after TPA injection. Into the control group, the same volume of PBS buffer was intraperitoneally injected, and the experiment was terminated 26 hours after TPA application.

26 hours after TPA application, the ear thickness was measured and photographs of both ears for each test group were taken in order to examine the degree of swelling and inflammation relief in the ears. After the test animal was sacrificed, the weight per area of the ear was measured, the ears of mice were fixed in 10% neutral buffered formalin at room temperature for 24 hours or more, and then embedded in paraffin to prepare 5 μm-thick sections, and the sections were stained with hematoxylin and eosin (H&E). The degrees of swelling of the ear and inflammatory cell infiltration were examined by observing the H&E-stained tissue section under an optical microscope.

### Example 6-2: Experimental Results

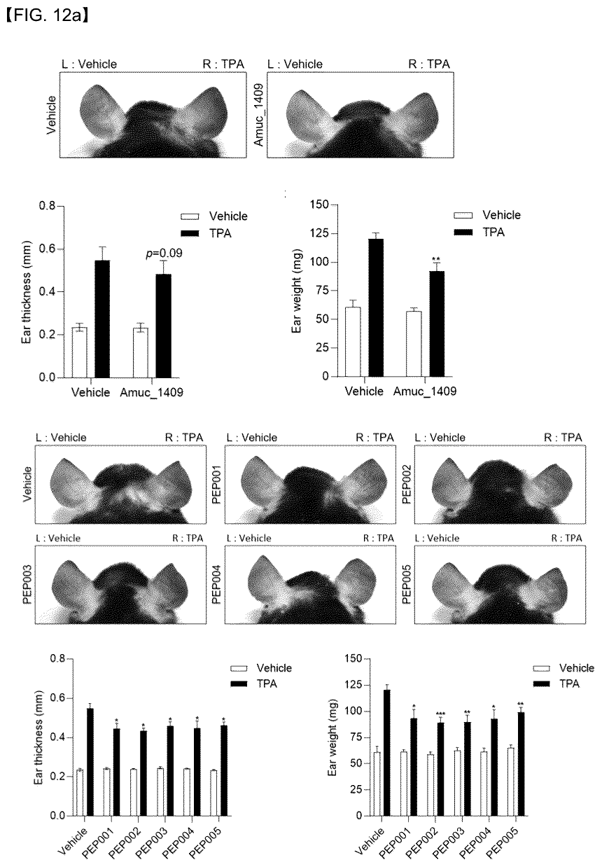

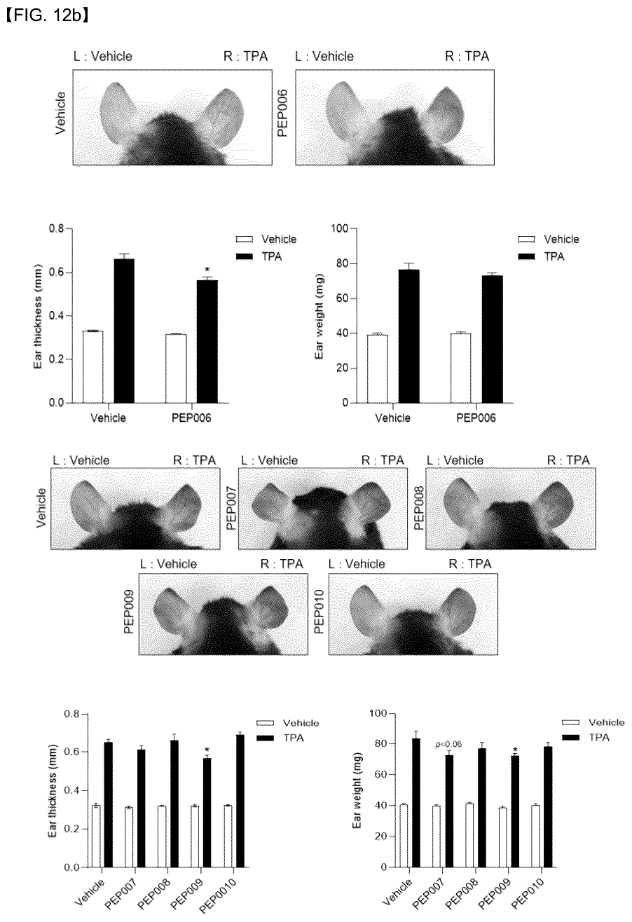

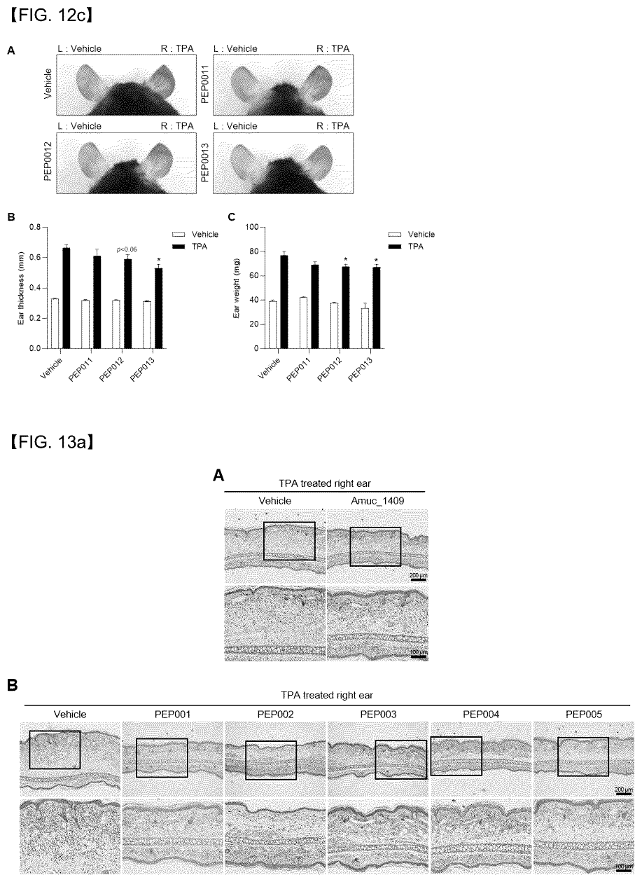

As a result of taking a macro photograph of the ear 26 hours after TPA application in a dermatitis model, it was confirmed that erythema and swelling of the ear were reduced overall in the group administered with Amuc_1409 compared to the group administered with PBS buffer. As a result of measuring the ear thickness, ear thickness tended to decrease in the group administered with Amuc_1409 compared to the group administered with PBS buffer. In addition, as a result of measuring the ear weight, the ear weight significantly decreased in the group administered with Amuc_1409 compared to the group administered with PBS buffer (p<0.05, Student's t-test) (FIG. 12a).

As a result of taking a macro photograph of the ear 26 hours after TPA application in the groups administered with PEP001, PEP002, PEP003, PEP004, and PEP005, it was confirmed that erythema and swelling of the ear were significantly reduced in the groups administered with PEP001, PEP002, PEP003, PEP004, and PEP005 compared to the group administered with PBS buffer. As a result of measuring the ear thickness, ear thickness significantly decreased in the groups administered with PEP001, PEP002, PEP003, PEP004, and PEP005 compared to the group administered with PBS buffer. In addition, as a result of measuring the ear weight, the ear weight significantly decreased in the groups administered with PEP001, PEP002, PEP003, PEP004, and PEP005 compared to the group administered with PBS buffer (p<0.05, Student's t-test) (FIG. 12a). In the groups administered with PEP006 to PEP013 as well, it was confirmed that erythema and swelling of the ear were reduced overall, and the ear thickness and ear weight also tended to decrease (FIGS. 12b and 12c).

In addition, as a result of examining histopathological findings in the mouse ear tissue through H&E staining, ear tissue swelling and inflammatory cell infiltration caused by TPA application were significantly reduced in the groups administered with Amuc_1409 and PEP001 to PEP013 compared to the group administered with PBS buffer (FIGS. 13a and 13b).

Through this, it has been confirmed that the peptides of the present invention have an effect of preventing and treating inflammatory skin diseases.

### Example 7: Verification of Efficacy of Novel Peptides in Animal Model of Depression and Anxiety Disorder

### Example 7-1: Experimental Method

By Daehan Biolink, 7-week-old male C57BL/6J mice weighing 20-22 g were supplied, and the animals were sufficiently supplied with solid feed and water until the day of the experiment, acclimatized for 1 week while maintaining an environment with a temperature of 22° C. 2° C., a humidity of 55%±15%, and a 12-hour light/dark cycle, and then used in the experiment.

From 3 days before inducing depression and anxiety, the peptide was dissolved in PBS buffer and orally administered to the mice by 150 μL per animal at the same time once a day. Amuc_1409 or PEP001, PEP002, PEP003, PEP004, or PEP005 was dissolved in PBS buffer at a concentration of 6.7 μM and then used, and novel tetrapeptides PEP006, PEP007, PEP008, PEP009, and PEP010 and novel tripeptides PEP011, PEP012, and PEP013 were each dissolved in PBS buffer at a concentration of 0.2 μg/150 μL and then used.

On the 3rd day of peptide administration, lipopolysaccharide (LPS) dissolved in PBS buffer was intraperitoneally injected at a concentration of 0.8 mg/kg to induce depression and anxiety.

Tail suspension test (TST) and forced swim test (FST) were conducted to analyze the effect on depression. As the TST, a tail suspension device was installed in an open white acrylic box of 20 cm×25 cm×30 cm size, and the mouse was suspended upside down at a height where the mouse's tail would not touch the floor, and then the time for which the mouse maintained immobility while observing in the same environment was measured for 6 minutes. FST is to confirm the degree of despair, a depressive symptom by measuring the immobility of mice in a cylinder at a depth where the paws and tail do not touch the bottom. Water at around 25° C. was filled in a transparent acrylic cylinder (45 cm depth×18 cm diameter) up to 30 cm from the bottom, the mouse was exposed to water, and the time for which the mouse maintained immobility while swimming was measured for 6 minutes. For FST and TST analysis, out of a total of 6 minutes of photographing, 2 minutes after the start was regarded as adaptation time, and the total time for which the mouse did not move was measured and recorded for 4 minutes before the end.

An elevated plus-maze (EPM) test was performed to analyze the effect on anxiety level. The EPM device consists of two open arms (50 cm×10 cm) and two crossed arms (50 cm×10 cm) at a height of 50 cm from the floor, and the arms are connected by a central area of 10 cm×10 cm. At the start of the experiment, the mouse was placed in the center of the maze and the time for which the mouse stayed in the open arm was measured for a total of 5 minutes using a video camera, and the percentage of time spent in the open arm was calculated and recorded [(time spent in open arm/time spent in open and closed arms)×100].

In addition, in order to examine the anti-inflammatory activity according to the administration of Amuc_1409 and PEP001, PEP002, PEP003, PEP004, and PEP005 in an animal model of depression and anxiety, the expression of Ifnγ and Il-10 mRNA, which are major inflammatory genes related to depression, in brain tissue (hippocampus) was examined.

### Example 7-2: Experimental Results

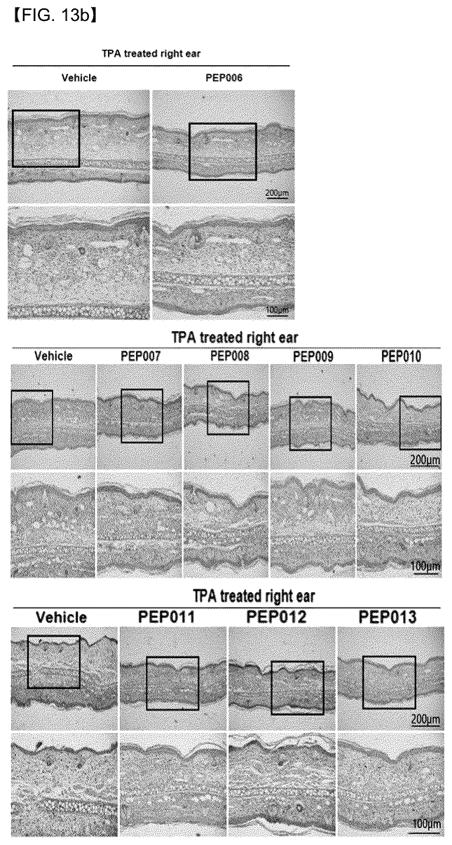

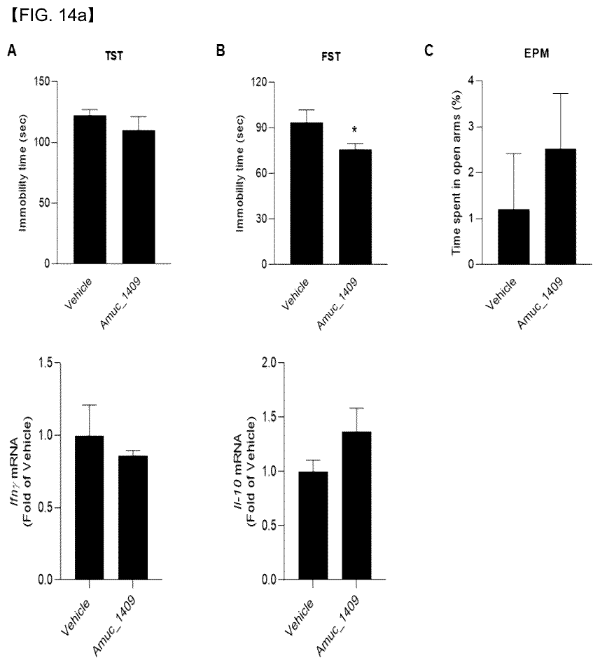

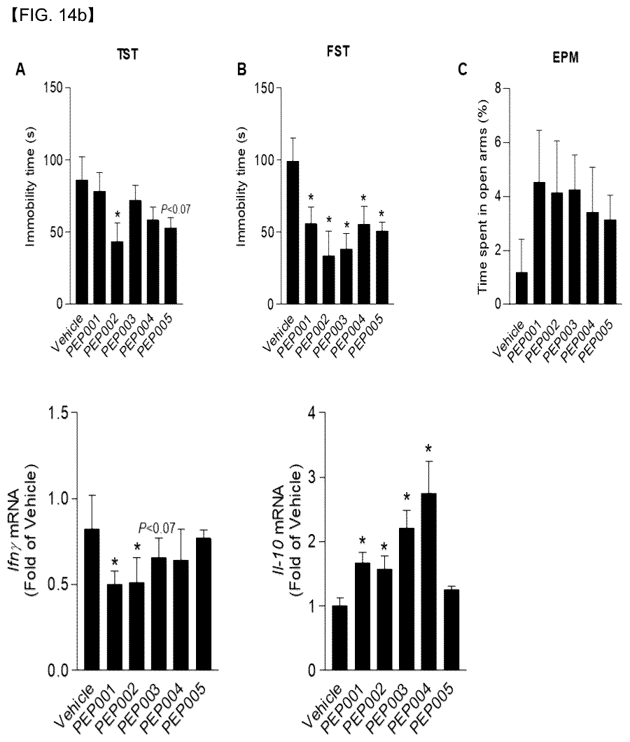

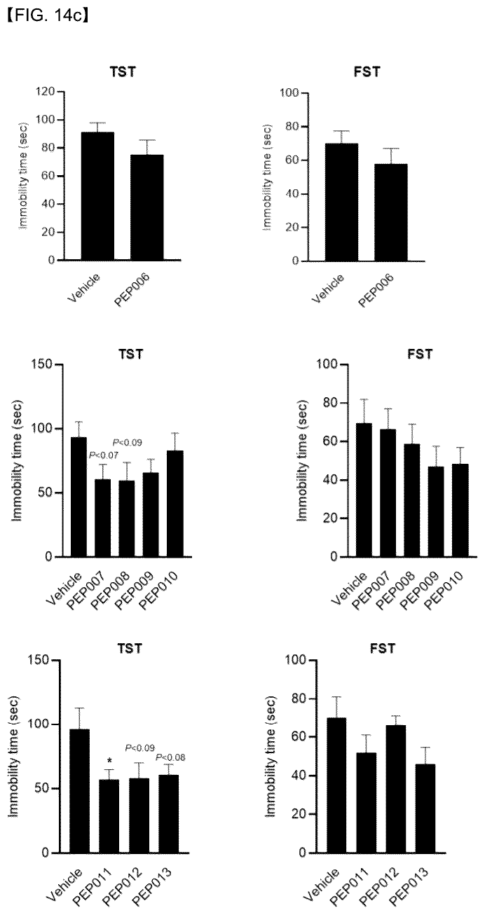

In the tail suspension and forced swim tests in a depression and anxiety model, the time for which the mouse maintained immobility due to tail suspension and forced swim significantly decreased in the group administered with Amuc_1409 compared to the group administered with PBS buffer. In addition, in the elevated plus-maze test, the time for which the mouse spent in the open arm increased in the group administered with Amuc_1409 compared to the group administered with PBS buffer, indicating anxiolytic efficacy (p<0.05, Student's t-test) (FIG. 14a).

As a result of analyzing the expression of inflammation-related genes in brain tissue (hippocampus) through RT-qPCR, the inflammatory cytokine Ifnγ mRNA expression decreased but the anti-inflammatory cytokine Il-10 mRNA expression increased in the group administered with Amuc_1409 compared to the group administered with PBS buffer (FIG. 14a).

In the tail suspension and forced swim tests in a depression and anxiety model, the time for which the mouse maintained immobility due to tail suspension and forced swim significantly decreased in the groups administered with PEP001, PEP002, PEP003, PEP004, and PEP005 compared to the group administered with PBS buffer (p<0.05, Student's t-test). In addition, in the elevated plus-maze test, the time for which the mouse spent in the open arm increased in the groups administered with PEP001, PEP002, PEP003, PEP004, and PEP005 compared to the group administered with PBS buffer, indicating anxiolytic efficacy (p<0.05, Student's t-test) (FIG. 14b).

As a result of analyzing the expression of inflammation-related genes in brain tissue (hippocampus) through RT-qPCR, the inflammatory cytokine Ifnγ mRNA expression significantly decreased but the anti-inflammatory cytokine Il-10 mRNA expression significantly increased in the groups administered with PEP001, PEP002, PEP003, PEP004, and PEP005 compared to the group administered with PBS buffer (FIG. 14b).

In the tail suspension test, the immobility time in the inverted state with the tail suspended decreased in the groups administered with the novel tetrapeptides PEP006, PEP007, PEP008, PEP009, and PEP010 and novel tripeptides PEP011, PEP012, and PEP013 compared to the group administered with PBS buffer. In the forced swim test, the immobility time in the inverted state with the tail suspended tended to decrease in the groups administered with the novel tetrapeptides PEP006, PEP007, PEP008, PEP009, and PEP010 and novel tripeptides PEP011, PEP012, and PEP013 compared to the group administered with PBS buffer (FIG. 14c).

Through this, it can be seen that administration of the peptides of the present invention is effective in preventing and treating depression and anxiety disorder.

### Example 8: Verification of Efficacy of Novel Peptides in Animal Model of Cognitive/Memory Disorder

### Example 8-1: Verification of Efficacy of Novel Peptides in Animal Model of Alzheimer's Disease

To 5-month-old APP/PS1 Alzheimer's disease male mice with amyloid deposition and cognitive and memory disorder in the brain, Amuc_1409 was orally administered to the mice by 5 μg daily 5 days a week for 8 weeks. PEP001 was dissolved in PBS buffer at a concentration of 2.4 μM, and then orally administered to the mice by 150 μL per animal 5 days a week for 8 weeks. To the control group (vehicle), PBS was orally administered in the same volume. In addition, normal mice (Non-Tg) not administered with the novel protein fragments were used as a control group.

In order to test the effect of improving cognitive function in APP/PS1 Alzheimer's disease mice administered with Amuc_1409 and PEP001, a novel object recognition test was performed. When the object seen 24 hours ago and a novel object were provided, the exploration time and the number of explorations for the novel object were measured to test cognition and memory.

As a result of the analysis, it was confirmed that APP/PS1 Alzheimer's disease mice had significantly decreased cognition for novel objects and memory compared to normal mice, and were unable to distinguish between novel objects and familiar objects, but it was confirmed that cognition and memory were significantly improved when Amuc_1409 and PEP001 were administered for 8 weeks (p<0.05, Student's t-test) (FIG. 15).

### Example 8-2: Verification of Efficacy of Novel Peptides in Animal Model of Cognitive/Memory Disorder

LPS was intraperitoneally injected to C57BL/6J male mice at 250 μg/kg/day daily for 7 days to induce cognitive and memory disorders. Amuc_1409, a protein derived from Akkermansia muciniphila, was orally administered to the mice by 5 μg daily from one week before LPS administration until the cognitive and memory test was performed. PEP002, PEP003, PEP004, or PEP005 was dissolved in PBS buffer at a concentration of 2.4 μM and then orally administered to the mice by 150 μL per animal daily at the same time once a day from 3 days before LPS administration until the cognitive and memory test was performed. To the control group (vehicle), PBS was orally administered in the same volume.

In order to test the effect of improving cognitive function in mice with cognitive and memory disorder administered with Amuc_1409 or PEP002, PEP003, PEP004, or PEP005, a novel object recognition test was performed. When the object seen 24 hours ago and a novel object were provided, the exploration time and the number of explorations for the novel object were measured to test cognition and memory.

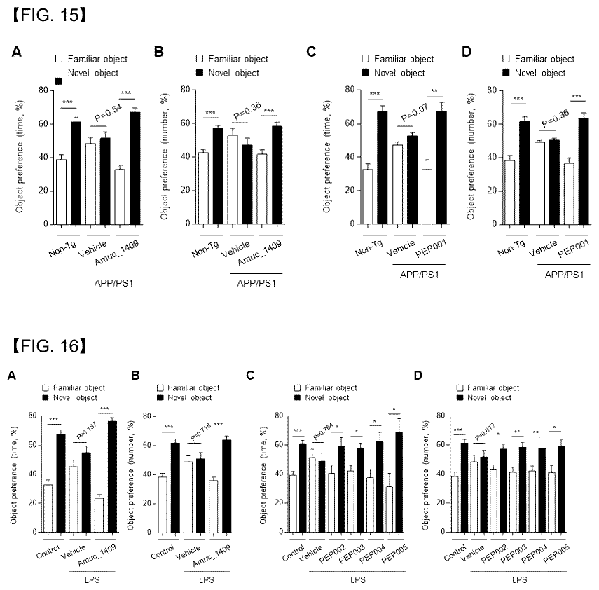

As a result of the analysis, it was confirmed that the cognition for novel objects and memory were significantly reduced and it was difficult to distinguish between novel objects and familiar objects when LPS was administered to the mice, but it was confirmed that cognitive and memory impairments caused by inflammatory reactions following LPS administration were suppressed and memory and cognitive function were improved when Amuc_1409 or PEP002, PEP003, PEP004, and PEP005 were administered to the mice compared to the control group (p<0.05, Student's t-test) (FIG. 16).

### Example 8-3: Verification of Cognitive/Memory Efficacy of Novel Peptides in Naturally Aged Mice

An experiment was conducted to verify the efficacy of the peptides of the present invention in an animal model of cognitive/memory disorder due to natural aging.

Amuc_1409 was orally administered to 16.5-month-old male mice by 4.5 μg daily 5 days a week for 8 weeks, and PEP002, PEP003, PEP004, or PEP005 was dissolved in PBS buffer at a concentration of 2.4 μM and then orally administered to 16-month-old male mice by 150 μL per animal at the same time once a day for 3 weeks. To the control group (vehicle), PBS was orally administered in the same volume.

In the case of the groups administered with the novel tetrapeptides and tripeptides (PEP006 to PEP013), each peptide was orally administered to 19-month-old female C57BL6/J mice by 0.2 μg daily 5 days a week for 8 weeks. At this time, the peptide was dissolved in PBS buffer and orally administered to the mice at 0.2 μg/150 μL per animal at the same time once a day. To the vehicle group, PBS was orally administered in the same volume. At this time, 4-month-old female C57BL6/J mice were used as a young control group.

A novel object recognition test (NORT) was performed to test the effect of improving cognitive function in naturally aged mice. When the object seen 24 hours ago and a novel object were provided, the exploration time and the number of explorations for the novel object were measured to test cognition and memory. In addition, a Y-maze alteration test was performed to confirm the effect of enhancing spatial perception and memory. In the Y-maze alteration test, spontaneous alteration (%) was evaluated by measuring the order of entering each Y-shaped maze (A, B and C). A score of 1 (actual alteration: order of ABC, BCA, CAB, or the like) was given if the mouse entered the three different regions sequentially, and a score was not given if the mouse did not enter consecutively. The spontaneous alteration was calculated and expressed as the total number of alterations/(total number of entries−2)×100.

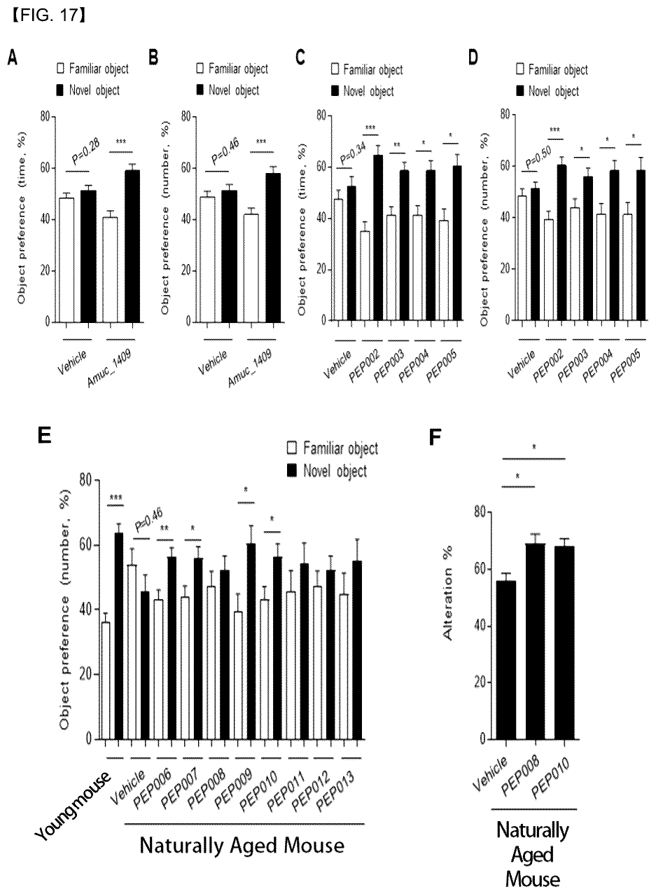

As a result of the analysis, it was confirmed that naturally aged mice had remarkably decreased cognition for novel objects and memory and were unable to distinguish between novel objects and familiar objects, but it was confirmed that cognition and memory were significantly improved or tended to be improved compared to the control group when Amuc_1409 and PEP002 to PEP013 were administered (p<0.05, Student's t-test). In addition, as a result of Y-maze alteration test analysis, it was confirmed that the administration of the novel tetrapeptides PEP008 and PEP010 significantly improved the spontaneous alteration compared to naturally aged mice administered with PBS buffer (FIG. 17).

Through this, it has been confirmed that the peptides of the present invention have effects of preventing and treating cognitive and memory disorders as well as preventing and treating degenerative brain diseases, and have an effect of improving cognition and memory.

### Example 9: Verification of Efficacy of Novel Peptides in Animal Model of Developmental Disability

In order to verify the efficacy of the peptides of the present invention on developmental disability, BTBR mice (BTBR T⁺ltpr3^{tf}/J (BTBR)), a developmental disability model, were used as an experimental animal.

To 3-week-old male BTBR mice, Amuc_1409 was orally administered by 5 μg daily 5 days a week, and PBS (phosphate-buffered saline) was administered to the control group. PEP001, PEP002, PEP003, PEP004, and PEP005 were each dissolved in PBS buffer at a concentration of 2.4 μM and orally administered to the mice by 150 μL per animal at the same time once a day 5 days a week, and novel tetrapeptides PEP006, PEP007, PEP008, PEP009, and PEP010 and novel tripeptides PEP011 and PEP013 were each dissolved in PBS buffer at 0.2 μg/150 μL and orally administered to the mice by 150 μL per animal at the same time once a day 5 days a week. After 4 weeks of administration, behavioral evaluation was performed by observing the social behavior of the mice. The mice were allowed to freely consume sterilized feed and water in a breeding facility in a specific pathogen free environment where the temperature was maintained at 22° C. to 24° C., and were bred while maintaining a 12-hour day/night cycle.

In order to analyze the social behavior of mice with developmental disability, the sniffing behavior with other mice was observed for 3 minutes, and the sniffing time and frequency were measured. It was confirmed that the social behavior of BTBR mice orally administered with Amuc_1409 and PEP001 to PEP013 significantly increased or tended to increase compared to the group of BTBR mice administered with vehicle (p<0.05, Student's t-test) (FIG. 18).

Through the results, it has been confirmed that the administration of Amuc_1409 and PEP001, PEP002, PEP003, PEP004, and PEP005 is effective in preventing and treating social behavior disorder indicated by developmental disability, and it can be seen that the peptides provided in the present invention are effective in preventing, treating, and improving developmental disability including autism spectrum disorder.

### Example 10: Verification of Tissue Regeneration Efficacy of Novel Peptides

In order to confirm the tissue regeneration efficacy of the novel peptides, an experiment was conducted using a wound-induced model induced by a full-thickness skin defect of mouse's tail.

For a full-thickness skin defect wound-induced model, 9-week-old specific pathogen free (SPF) male C57BL/6J mice were anesthetized, and then in order to indicate full-thickness wounding, a full-thickness wound with a size of 10 mm×3 mm was made on the dorsal part of the mouse's tail, about 1.0 cm away from the base of the mouse's tail, using an aseptic dissection mass No. 10. Bleeding was stopped by compressing the wound, and the wound was covered with a film spray dressing (Cavilon, 3M).

For 28 days from the day of wound induction, Amuc_1409 or PEP001 to PEP013 were each dissolved in vehicle (50% ethanol, 20% propylene glycol, 30% water) at a concentration of 120 μM and then applied to the wound site by 25 μL per animal once a day. For the control group, the same volume of vehicle (50% ethanol, 20% propylene glycol, 30% water) was applied to the wound site. The test was terminated on day 29 after wound induction.

In order to observe the wound healing effect, the wound sites of animals in each test group were individually photographed using a digital camera at a constant height from the wound for 28 days at 7-day intervals from the day of wound induction, and the wound area was measured using the Image J Soft program.

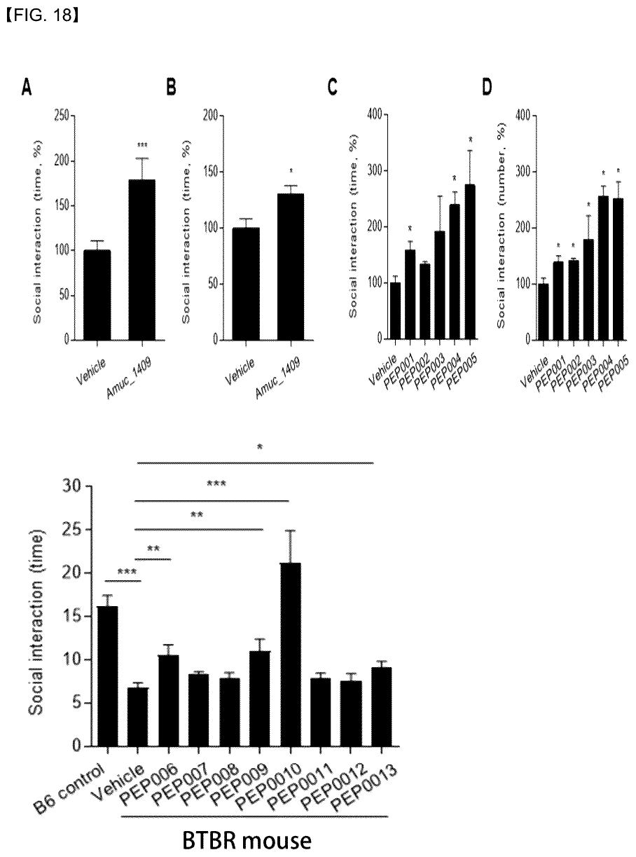

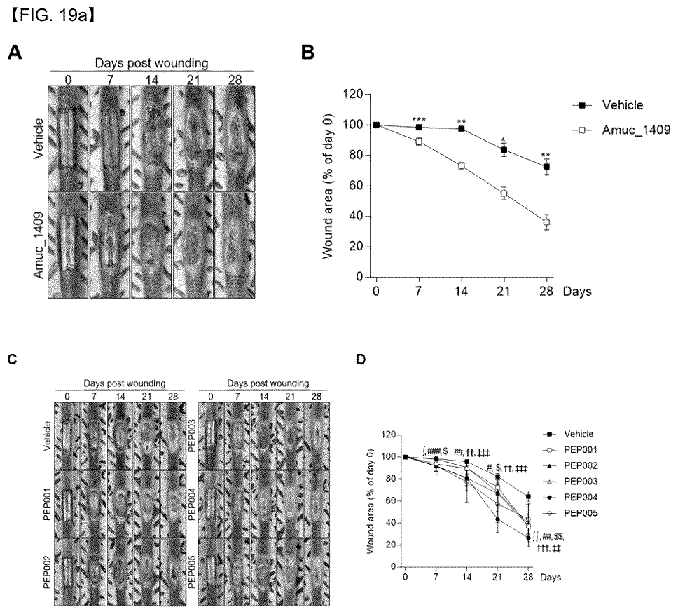

As a result of the analysis, in the full-thickness skin defect wound-induced animal model, it was visually confirmed that the wound size rapidly decreased in the groups to which Amuc_1409 and PEP001, PEP002, PEP003, PEP004, and PEP005, novel tetrapeptides PEP006, PEP007, PEP008, PEP009, and PEP010, and novel tripeptides PEP011 and PEP013 were applied compared to the group to which the vehicle was applied. In addition, as a result of measuring the wound area, the wound area significantly decreased in the groups to which Amuc_1409 and PEP001, PEP002, PEP003, PEP004, PEP005, PEP006, PEP007, PEP008, PEP009, and PEP010 and novel tripeptides PEP011 and PEP013 were applied compared to the group to which the vehicle was applied (p<0.05, Student's t-test) (FIGS. 19a and 19b).

Through this, it has been confirmed that the peptides of the present invention have efficacy on damage recovery and regeneration of body tissues including skin tissues.

### Example 11: Verification of Tissue Regeneration Promoting Efficacy of Novel Peptides

In order to confirm that the novel peptides of the present invention have tissue regeneration promoting efficacy, it was examined whether muscle tissue regeneration was promoted and muscle strength was improved.

### Example 11-1. Verification of Muscle Strength Improvement and Muscle Regeneration Efficacy of New Peptides in Animal Model of Aging

The efficacy of the peptides was verified using an animal model of muscle atrophy/muscle strength loss due to natural aging (geriatric sarcopenia).

At the time of administration of Amuc_1409, 80 to 83-week old C57BL/6 male mice were used. Amuc_1409 was dissolved in PBS buffer at a concentration of 2.4 μM and then orally administered to the mice by 150 μL per animal at the same time once a day for 32 weeks. To the control group, PBS buffer was orally administered in the same manner.

At the 16th week of Amuc_1409 administration, aged mice were allowed to hold the wire net connected to the muscle strength probe of the grip strength meter with all four paws, and then the tail was carefully pulled backward to measure the maximum grip strength at the moment at which the mice held onto the wire net. For muscle weight, after administration, the gastrocnemius (GC), soleus, and tibialis anterior (TA) muscles from both hind paws of the aged mice of each group were carefully separated to avoid damage, and each weight was measured. Muscle weight versus body weight was calculated by correcting with the body weight of the mouse.

After the end of the experiment, in order to analyze the size of muscle fibers in each group, TA muscles were fixed in 10% formalin, and frozen section samples (8 μm) were prepared to prepare slides. The prepared slides were washed with PBS buffer solution, permeabilized with 0.5% Triton X-100, blocked, and treated with primary antibody (anti-laminin). After 24 hours, a fluorescent secondary antibody (Alexa Fluor 488 Goat anti-rabbit IgG) was applied, and the slides were finally treated with DAPI for nuclear staining. The immunostained slides were subjected to photographing of muscle tissue using a confocal microscope. Using image analysis software (ImageInside), the distribution of cross-sectional area of muscle fibers was calculated for each size from a minimum of 500 μm² or less to 3500 μm² or more.

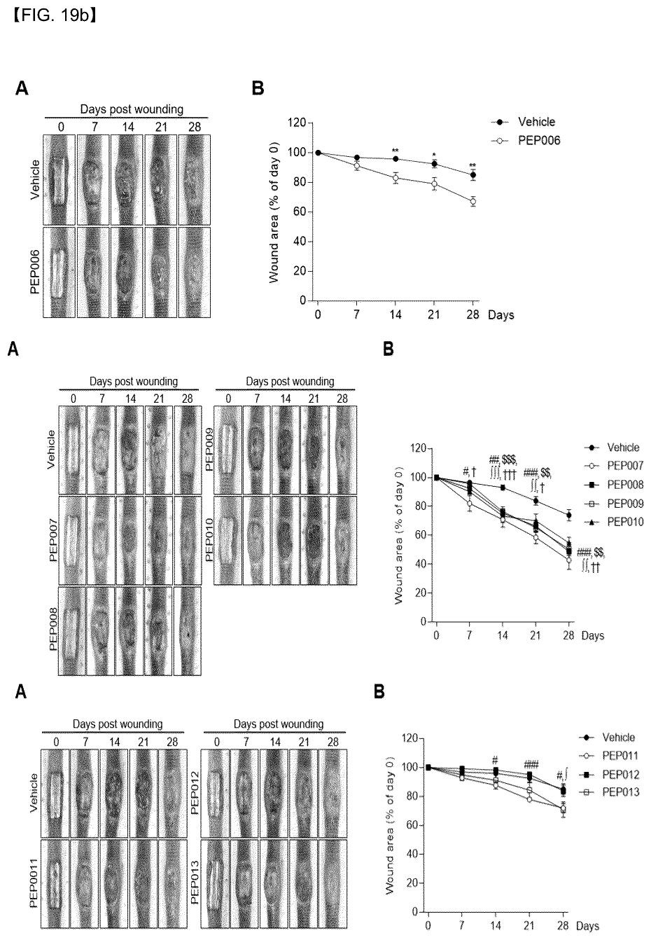

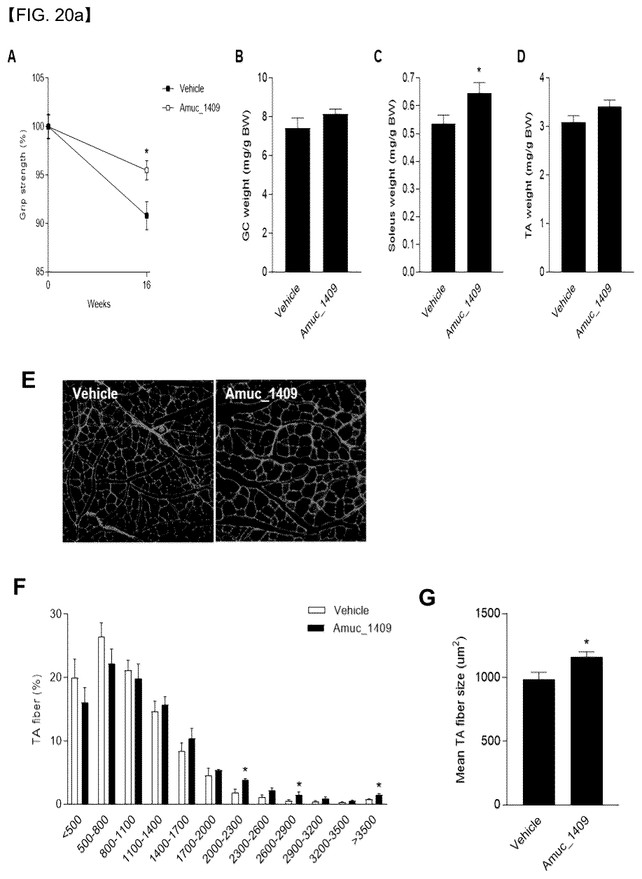

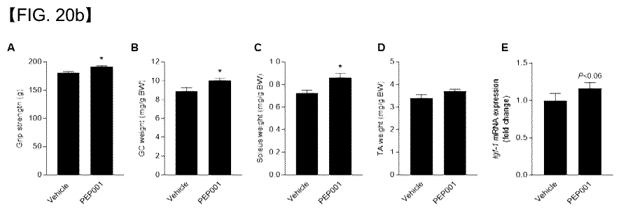

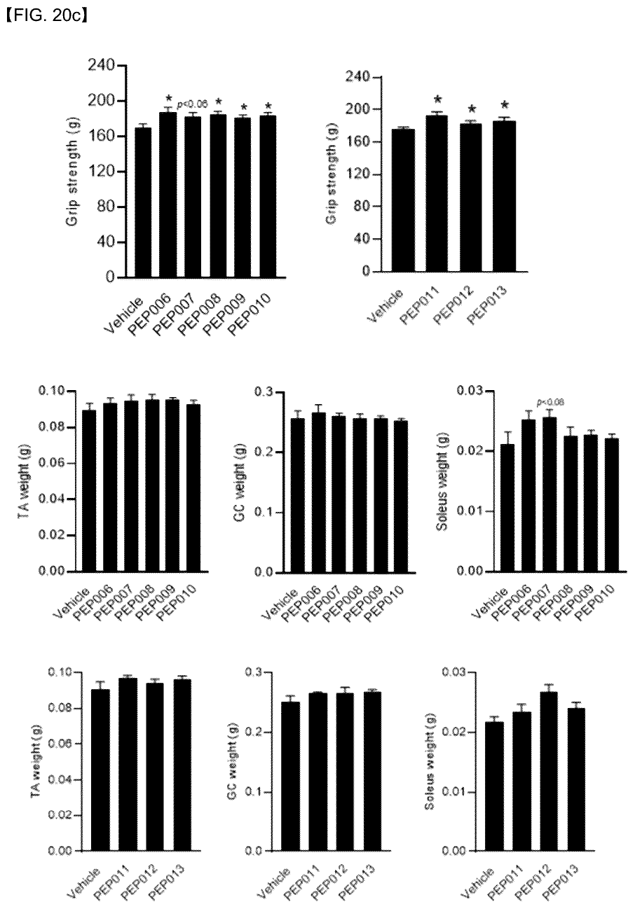

As a result of the analysis, in the muscle strength attenuation test, at the 16th week of Amuc_1409 administration, it was confirmed that muscle strength significantly increased in the group administered with Amuc_1409 compared to the grip strength of the group administered with PBS buffer (p<0.05, Student's t-test) (FIG. 20a). In addition, the GC, soleus, and TA muscle weights measured after the experiment were all increased in the group administered with Amuc_1409 compared to the group administered with PBS buffer, and the soleus muscle significantly increased, confirming that the decrease in muscle mass due to aging was suppressed (p<0.05, Student's t-test) (FIG. 20a).

In addition, in order to analyze the effect of Amuc_1409 administration on muscle atrophy due to aging, immunostaining was performed on laminin, one of the main components of the muscle cell basement membrane. As a result, it was observed that the number of muscle fibers with a cross-sectional size of 500 μm² or less in the TA muscle of mice administered with Amuc_1409 decreased and muscle fibers with a size of 2,000 μm² or more significantly increased compared to the group administered with PBS buffer (p<0.05, Student's t-test) (FIG. 20a). In addition, as a result of calculating the average size of all muscle fibers, including all of the small to large sizes, the average size of all muscle fibers in aged mice administered with Amuc_1409 significantly increased compared to the group administered with PBS buffer, confirming the effect of inhibiting or improving muscle fiber atrophy (p<0.05, Student's t-test) (FIG. 20a).

Meanwhile, in order to examine the efficacy of Amuc_1409 as well as its fragments, 82- to 83-week-old C57BL/6 male mice were used. PEP001 was dissolved in PBS buffer at a concentration of 2.4 μM and then orally administered to the mice by 150 μL per animal at the same time once a day for 10 weeks. To the control group, PBS buffer was orally administered in the same manner. At the 10th week of PEP001 administration, the maximum grip strength and muscle weight were calculated in the same manner as the measurement in Amuc_1409.

At the end of the experiment, a part of the GC muscle excised was homogenized with Trizol to extract RNA. Reverse transcriptase was added to the obtained RNA and reacted to synthesize cDNA, and SYBR Green and primers were added to perform quantitative real-time PCR (RT-qPCR). The gene expression level of insulin-like growth factor (IGF-1) involved in protein synthesis in muscle cells was then quantified.

As a result of the analysis, in the muscle strength attenuation test, at the 10th week of PEP001 administration, it was confirmed that muscle strength significantly increased in the group administered with PEP001 compared to the grip strength of the group administered with PBS buffer (p<0.05, Student's t-test) (FIG. 20b). In addition, the GC, soleus, and TA muscle weights measured after the experiment were all increased in the group administered with PEP001 compared to the group administered with PBS buffer, and the GC and soleus muscles significantly increased, confirming that the decrease in muscle mass due to aging was suppressed (p<0.05, Student's t-test) (FIG. 20b).

In the GC muscle excised at the 10th week of PEP001 administration, it was observed that protein synthesis in muscle cells was promoted and IGF-1 gene expression tended to increase compared to the group administered with PBS buffer (FIG. 20b).

In addition, in order to confirm the efficacy of the novel tetrapeptides and tripeptides PEP006 to PEP013, the improvement efficacy on geriatric sarcopenia was examined using naturally aged 76 to 78-week old female C57BL/6J mice. For an additional 12 weeks, the novel tetrapeptides PEP006, PEP007, PEP008, PEP009, and PEP010 or novel tripeptides PEP011, PEP012, and PEP013 were each dissolved in PBS buffer at a concentration of 0.2 μg/150 μL and then orally administered to the naturally aged mice by 150 μL per mouse at the same time until the end of the test. To the control group, PBS buffer was orally administered in the same manner.

For all animals, general symptoms were observed immediately before administration at the start of administration, and grip strength was measured at the 11th week of administration in order to analyze muscle strength attenuation. For the measurement of grip strength, mice were allowed to hold the wire net connected to the muscle strength probe of the grip strength meter with all four paws, and then the tail was carefully pulled backward to measure the maximum grip strength at the moment at which the mice held onto the wire net. At the end of the test, the tibialis anterior (TA), gastrocnemius (GC), and soleus muscles of both hind limbs of the mouse were separated and weighed.

As a result of the experiment, it was confirmed that grip strength significantly increased in the groups administered with PEP006, PEP008, PEP009, and PEP010 and increased to a significant level in the group administered with PEP007 compared to the group administered with PBS buffer (p<0.05, Student's t-test) (FIG. 20c). It was confirmed that grip strength significantly increased in also the groups administered with the novel tripeptides PEP011, PEP012, and PEP013 compared to the group administered with PBS buffer (p<0.05, Student's t-test) (FIG. 20c).

In addition, as a result of analyzing muscle weight to analyze the effect of administration of new tetrapeptides PEP006, PEP007, PEP008, PEP009, and PEP010 on muscle mass, TA and GC muscles tended to increase in the groups administered with PEP006, PEP007, PEP008, PEP009, and PEP010 compared to the group administered with PBS buffer. Soleus muscle increased to a significant level in the group administered with PEP007 and tended to increase in the groups administered with PEP006, PEP008, PEP009, and PEP010 compared to the group administered with PBS buffer. As a result of analyzing muscle weight to analyze the effect of administration of new tripeptides PEPO 11, PEP012, and PEP013 on muscle mass, TA, GC, and soleus muscles tended to increase in the groups administered with PEP011, PEP012, and PEP013 compared to the group administered with PBS buffer (FIG. 20c).

### Example 11-2. Verification of Muscle Regeneration Efficacy of Novel Peptides in Myoblast Model

Myoblast C2C12 (myoblast) was dispensed into a 6 well plate, and then cultured in a DMEM medium containing 10% FBS and 1% antibiotics at 37° C. in a 10% CO₂ incubator. Thereafter, in order to induce differentiation, the medium was replaced with a differentiation medium containing 2% horse serum, the C2C12 cells were treated with PEP004 and PEP005 at a concentration of 64 nM during the time for induction of differentiation, and formation of myotubes was observed.

After differentiation of myoblasts, the cells were treated with dexamethasone (25 μM), which causes muscle atrophy, and PEP004 and PEP005 simultaneously, and then RNA was extracted from the cells using Trizol. Reverse transcriptase was added to the obtained RNA and reacted to synthesize cDNA, and SYBR Green and primers were added to perform quantitative real-time PCR (RT-qPCR). Expression levels of Atrogin-1 and MuRF1, which are factors involved in muscle atrophy, were measured to determine whether atrophy of differentiated myoblasts was inhibited.

As a result of the analysis, the expression of Atrogin-1 and MuRF1, which are muscle protein degradation genes, significantly increased in cells treated with dexamethasone after induction of differentiation of C2C12 myoblasts, but the expression of the genes significantly decreased in cells treated with PEP004 and PEP005 together with dexamethasone, (p<0.05, Student's t-test) (FIG. 21).

Through this, it has been confirmed that the peptides of the present invention have an effect of promoting muscle regeneration and an effect of enhancing muscle strength and improving muscle strength.

### Example 12: Verification of Hair Regeneration and Hair Growth Promoting Efficacy of Novel Peptides

### Example 12-1: Experimental Method

In order to examine the hair growth promoting efficacy, 8-week-old C57BL/6 mice with telogen hair having pink dorsal skin were used. The mice were anesthetized, then the hair on the back of the mouse was removed using an electric hair clipper for animals. Amuc_1409 was dissolved in vehicle (50% ethanol, 20% propylene glycol, 30% water) at a concentration of 3.2 μM and then applied to the skin of the hair-removed site by 200 μL per animal using a pipette once a day for 21 days, and PEP006 to PEP013 were each dissolved in vehicle (50% ethanol, 20% propylene glycol, 30% water) at a concentration of 30 μM and then applied to the skin of the hair-removed site by 200 μL per animal using a pipette once a day for 28 days. For the control group, the same volume of vehicle (50% ethanol, 20% propylene glycol, 30% water) was applied to the skin.

In order to visually evaluate the hair growth promoting effect in each group, after anesthesia on day 21 after the start of the experiment, hair growth was observed and the hair growth site was photographed using a digital camera. Skin color and hair density were visually observed, scored according to the scoring criteria in Table 5, and graphed (Table 5).

TABLE 5
Scoring criteria for visual evaluation of hair growth

| Criteria | Score |
|---|---|
| Hair does not grow at all | 0 |
| Skin color darkens in less than 30% of epilated site | 1 |
| Hair does not grow in epilated site | |
| Skin color darkens in 30% to 70% of epilated site | 2 |
| Hair does not grow in the epilated site | |
| Skin color darkens in 70% or more of epilated site | 3 |
| Hair grows in less than 30% of epilated site | |
| Skin color darkens in 70% or more of epilated site | 4 |
| Hair grows in 30% to 70% of epilated site | |
| Skin color darkens in 70% or more of epilated site | 5 |
| Hair grows in 70% or more of epilated site | |
| Hair grows in 90% or more of epilated site | 6 |

After the experiment, the test animals were sacrificed, then the skin tissue of the mouse was fixed in 10% NBF at room temperature for 24 hours or more, and then embedded in paraffin to prepare a 5 μm-thick section, and the section was stained with (H&E) hematoxylin and eosin. The histological changes of the hair follicle tissue in the H&E-stained tissue sections were observed under an optical microscope. The number and depth of hair follicles and the thicknesses of dermis and subcutis were measured under an optical microscope, and the hair growth cycle was evaluated. The number of hair follicles was calculated by counting the number of hair follicles present in the subcutaneous tissue under a 100-magnification optical microscope. Changes in the depth of hair follicles and the thicknesses of dermis and subcutis were measured using the Image J program using a scale bar.

### Example 12-2: Experimental Results

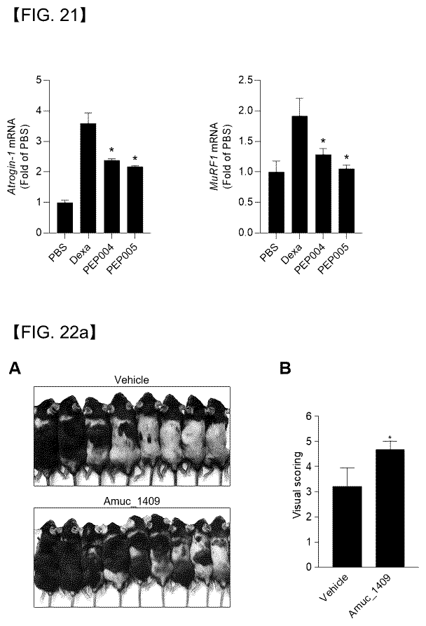

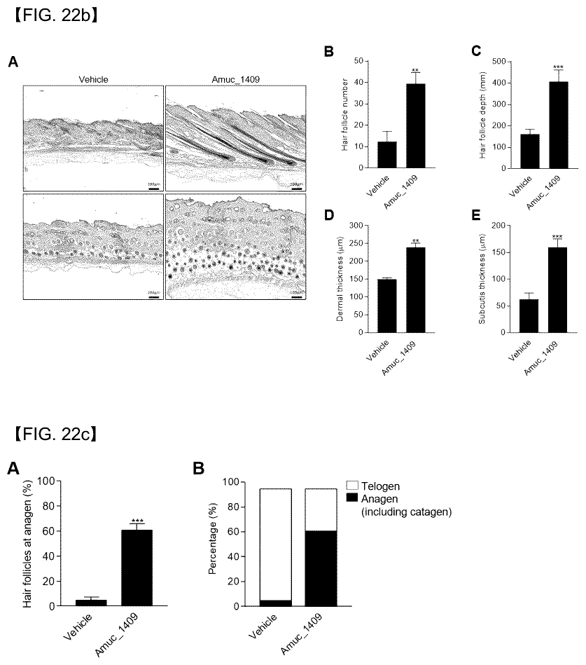

As a result of visually observing and scoring hair growth on day 21 after application, the proportion of the area where hair grew out to the total area significantly increased in the group to which Amuc_1409 was applied compared to the group to which vehicle was applied (p<0.05, Student's t-test) (FIG. 22a).

As a result of histological examination through H&E staining, the number of hair follicles present in the subcutaneous fat layer significantly increased and the depth of the hair follicle and the thicknesses of dermis and subcutis significantly increased in the group to which Amuc_1409 was applied compared to the group to which vehicle was applied (p<0.05, Student's t-test) (FIG. 22b).

In addition, as a result of analyzing the hair growth cycle histologically through H&E staining, the proportion of hair in the anagen phase significantly increased in the group to which Amuc_1409 was applied compared to the group to which vehicle was applied (p<0.05, Student's t-test) (FIG. 22c).

As a result of visually observing and scoring hair growth on day 28 after application, the proportion of the area where hair grew out to the total area tended to increase in the groups to which PEP006, PEP007, PEP008, PEP009, PEP011, PEP012, and PEP013 were applied compared to the group to which vehicle was applied (FIG. 22d).

Through this, it has been confirmed that the peptides of the present invention have hair regeneration and hair growth promoting efficacy.

### Example 13: Verification of Skin Regeneration Efficacy of Novel Peptides

An experiment was conducted to examine the skin regeneration ability of the peptides of the present invention. Human skin keratin cell line (HaCaT) was cultured in DMEM medium containing 10% fetal bovine serum (FBS) and 1% penicillin-streptomycin at 37° C. and 5% CO₂. When the cell density reached 80%, the cells were dispensed into a 6-well plate at 4×10⁵ cells per well and cultured for 24 hours. After 24 hours, the cells were treated with Amuc 1409 at a concentration of 30 nM for 1 hour, washed twice with PBS (phosphate buffered saline) after 1 hour, and irradiated with ultraviolet light at 10 mJ/cm² using an ultraviolet (UVB) irradiation device (CL-1000 UV crosslinker; UVP, USA). After UV irradiation, the medium was replaced with serum-free medium, and the cells were immediately treated with Amuc_1409 for 8 hours.

In relation to skin wrinkles due to skin aging, in order to examine the expression of MMP-1 and MMP-3 genes, which are collagenase, and Col1a1 and Col3a1 genes, which are collagen synthesis enzymes, RNA was isolated from the cells in each well using Trizol (Invitrogen, USA) and quantified by nanodrop, and then cDNA was synthesized using 1 μg of each RNA. A mixture obtained by adding MMP-1, MMP-3, Col1a1 and Col3a1 primers and fluorescent dye AccuPower® 2× GreenStar™ qPCR Master Mix (BIONEER, Korea) to the synthesized cDNA was subjected to real-time polymerase chain reaction in a real-time PCR machine.

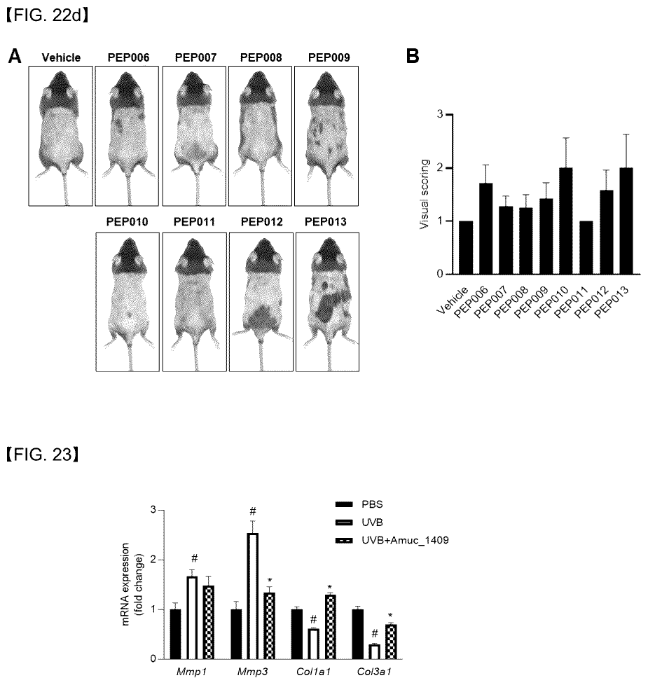

As a result of the analysis, when human skin keratinocytes were irradiated with UVB, the expression of MMP-1 and MMP-3 genes, which are collagenase, significantly increased compared to the group not irradiated with UVB but the expression of Coil a1 and Col3a1 genes, which are collagen synthesis enzymes, significantly decreased, confirming photoaging of skin cells. The expression of MMP-1 and MMP-3 genes in cells treated with Amuc_1409 significantly decreased compared to the control group irradiated with UVB, and the expression of Col1a1 and Col3a1 genes significantly increased, confirming that Amuc_1409 inhibits photoaging of skin cells by UVB irradiation (p<0.05, Student's t-test) (FIG. 23).

In addition, an experiment was conducted to verify the anti-skin aging efficacy in an animal model.

As an animal model of aging, 19- to 22-month old C57BL/6J naturally aged mice were used. Solid feed and water were sufficiently supplied until the day of the experiment, the environment was maintained at a temperature of 22° C. 2° C., humidity of 55%±15%, and a 12-hour light/dark cycle, and the mice were used for the experiment. Amuc_1409 and PEP001 were dissolved in PBS buffer at 10 μg/150 μL and 0.7 μg/150 μL, respectively, and then orally administered to the naturally aged mice by 150 μL per animal at the same time once a day for 20 weeks, and novel tetrapeptides PEP006, PEP007, PEP008, PEP009, and PEP010 and novel tripeptides PEP011, PEP012, and PEP013 were each dissolved in PBS buffer at a concentration of 0.2 μg/150 μL and then orally administered to the naturally aged mice by 150 μL per animal at the same time once a day for 10 weeks. To the control group, PBS buffer was orally administered in the same manner.

On the day to be the 10th week of administration, a part of the tissue was excised from the back of the mouse and fixed in 10% formalin for 24 hours or more for histopathological analysis.

The fixed skin tissue was embedded in paraffin to prepare a 5 μm-thick section, and the skin section was H&E-stained with hematoxylin and eosin to analyze the skin epidermis thickness and the number of hair follicles.

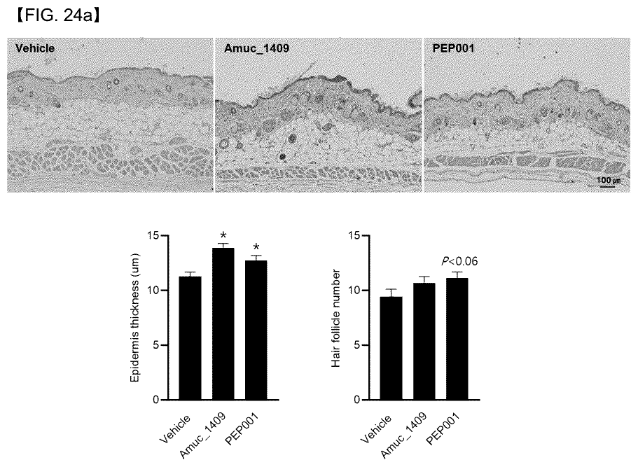

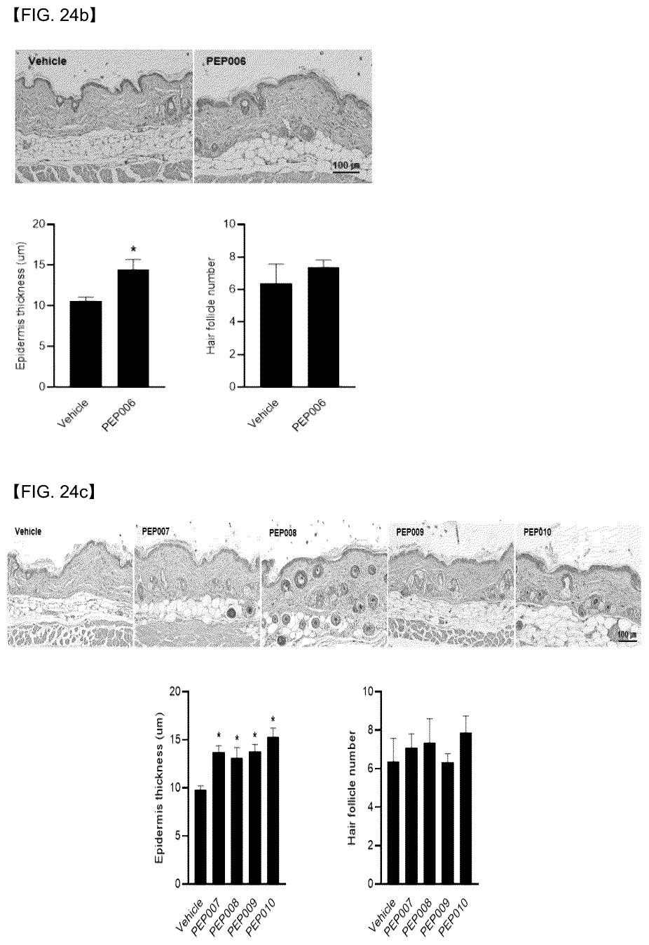

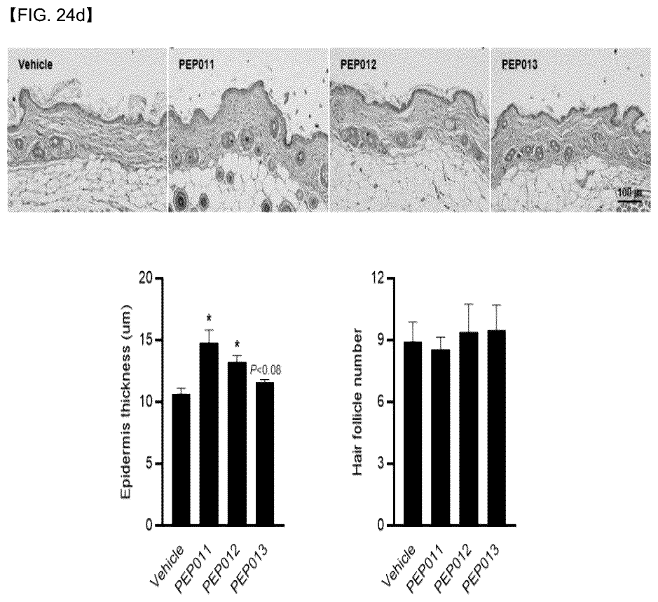

As a result of the analysis, when the thickness of the skin epidermis, which decreases as age increases in animal models of aging, was quantified, the thickness of the skin epidermis significantly increased in the groups administered with Amuc_1409 and PEP001, novel tetrapeptides PEP006, PEP007, PEP008, PEP009, and PEP010, and novel tripeptides PEP011 and PEP012 and increased close to a significant level by administration of PEP013 compared to the group administered with PBS buffer (p<0.05, Student's t-test). In addition, as age increases in animal models of aging, atrophy and fibrosis of hair follicles intensify and the number of hair follicles decreases overall, but and the number of hair follicles tended to increase in the groups administered with the novel tetrapeptides PEP006, PEP007, PEP008, PEP009, and PEP010, and novel tripeptides PEP012 and PEP013 compared to the group administered with PBS buffer (FIGS. 24a, 24b, 24c and 24d).

Through this, it has been confirmed that the peptides of the present invention have an effect of suppressing symptoms related to skin aging and promoting skin regeneration.

From the above description, those skilled in the art to which the present invention pertains will be able to understand that the present invention may be embodied in other specific forms without changing the technical spirit or essential characteristics thereof. In this regard, it should be understood that the embodiments described above are illustrative in all respects and not limiting. The scope of the present invention should be construed as including all changes or modifications derived from the meaning and scope of the following claims and their equivalent concepts rather than the detailed description above.

1. A peptide comprising a sequence represented by the following Formula 1 or 2: 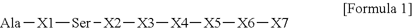
where, X1 is an amino acid classified as a nonpolar, basic, or polar amino acid;
X2 is Ser or absent;
X3 is Ile or absent;
X4 is Lys or absent;
X5 is Gly or absent;
X6 is Ala or absent; and
X7 is Tyr or absent, 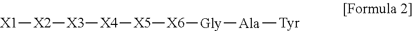
where X1 is Ala or absent;
X2 is Val or absent;
X3 is Ser or absent;
X4 is Ser or absent;
X5 is Ile or absent; and
X6 is an amino acid classified as a nonpolar, acidic, basic, or polar amino acid or absent.

2. The peptide according to claim 1, wherein the peptide comprises a sequence represented by the following Formula 1 or 2: 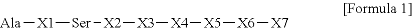
where X1 is Val, Gln, Gly, or Ile;
X2 is Ser or absent;
X3 is Ile or absent;
X4 is Lys or absent;
X5 is Gly or absent;
X6 is Ala or absent; and
X7 is Tyr or absent, 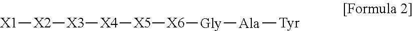
where X1 is Ala or absent;
X2 is Val or absent;
X3 is Ser or absent;
X4 is Ser or absent;
X5 is Ile or absent; and
X6 is Lys, Ala, Arg, Ile, Ser, Val, or absent.

3. The peptide according to claim 1, wherein the peptide comprises any one of amino acid sequences of SEQ ID NOs: 1 to 15.

4. A pharmaceutical composition for preventing or treating an inflammatory disease, the pharmaceutical composition comprising the peptide according to claim 1.

5. The pharmaceutical composition for preventing or treating an inflammatory disease according to claim 4, wherein the inflammatory disease is selected from the group consisting of arthritis, metabolic diseases, hepatitis, enteritis, gastritis, gastric ulcer, esophagitis, dermatitis, encephalitis, depression, anxiety disorder, cognitive disorder, memory disorder, degenerative brain disease, and developmental disability.

6. A composition for tissue regeneration comprising the peptide according to claim 1.

7. The composition for tissue regeneration according to claim 6, wherein the tissue is selected from skin, muscle, hair, hair follicles, or hair roots.

8. The composition for tissue regeneration according to claim 6, wherein the tissue regeneration is selected from skin regeneration, muscle tissue regeneration, wound healing, muscle strength enhancement, hair loss prevention, hair growth promotion, trichogen promotion, damaged hair improvement, or hair regeneration.

9. A composition for preventing or improving an inflammatory disease, the composition comprising the peptide according to claim 1, wherein
the composition is a health functional food, feed, veterinary medicine, quasi-drug, or cosmetic composition.

10. A composition for tissue regeneration comprising the peptide according to claim 1, wherein
the composition is a health functional food, feed, veterinary medicine, quasi-drug, or cosmetic composition.

11. A pharmaceutical composition for preventing or treating a tissue damage-related disease, the pharmaceutical composition comprising the peptide according to claim 1.

12. A method for preventing or treating an inflammatory disease, the method comprising administering the peptide according to claim 1 to a subject.

13. A tissue regeneration method comprising administering the peptide according to claim 1 to a subject.

14. A polynucleotide encoding the peptide according to claim 1.

15. A vector comprising the polynucleotide according to claim 14.

16. A pharmaceutical composition for preventing or treating an inflammatory disease, the pharmaceutical composition comprising the peptide according to claim 2.

17. A pharmaceutical composition for preventing or treating an inflammatory disease, the pharmaceutical composition comprising the peptide according to claim 3.
